(19) United States
(12) Patent Application Publication
(10) Pub. No.: US 2012/0058194 A1
(43) Pub. Date: Mar. 8, 2012

(54) PHARMACEUTICAL FORMULATIONS COMPRISING SUBSTITUTED BENZIMIDAZOLE DERIVATIVES

(76) Inventors: Navin Vaya, Hyderabad (IN); Mamta Mishra, Hyderabad (IN); Atul Shivaji Shinde, Nasik (IN); Harshal Prabhakar Bhagwatwar, Hyderabad (IN); Debashis Dash, Hyderabad (IN); Deepti Jain, Hyderabad (IN); Rahul Sudhakar Gawande, Hyderabad (IN); Vishal Lad, Hyderabad (IN); Sushant Dube, Hyderabad (IN); Venkateswarlu Vobalaboina, Hyderabad (IN); Srinivas Irukulla, Hyderabad (IN); Manikandan Ramalingam, Kanchaepuram (IN)

(21) Appl. No.: 13/217,901

(22) Filed: Aug. 25, 2011

Related U.S. Application Data
(60) Provisional application No. 61/393,389, filed on Oct. 15, 2010.
(60) Provisional application No. 61/416,994, filed on Nov. 24, 2010.
(60) Provisional application No. 61/446,114, filed on Feb. 24, 2011.

(30) Foreign Application Priority Data
Aug. 27, 2010 (IN) 2475/CHE/2010
Oct. 11, 2010 (IN) 3009/CHE/2010
Jan. 11, 2011 (IN) 94/CHE/2011

(51) Int. Cl. A61K 9/14; A61K 47/38; B05D 5/00; A61K 47/44; A61K 31/4439; A61P 1/04; A61K 47/36; A61K 47/46

(52) U.S. Cl. 424/494; 424/497; 424/493; 424/496; 424/498; 514/338; 427/2.21

(57) ABSTRACT

Stabilized substituted benzimidazole modified release pharmaceutical formulations with at least two drug-containing fractions, wherein the release from a first fraction precedes the release from a second fraction, pharmaceutical excipients, processes for preparing the stable formulations, packaging therefor, and their use in treatment of erosive esophagitis and heartburn associated with non-erosive gastroesophageal reflux disease.

### INTRODUCTION

Aspects of the present disclosure relate to formulations comprising at least one substituted benzimidazole derivative, and processes for preparing the same. In particular embodiments, the present disclosure relates to modified release formulations. In embodiments, formulations comprise a single enantiomer of lansoprazole which is dexlansoprazole, and processes for preparing the same. Aspects of the disclosure further relate to therapeutic uses and methods of treatment employing formulations comprising a substituted benzimidazole drug or a single enantiomer thereof.

Several substituted benzimidazole derivatives, including rabeprazole, omeprazole, esomeprazole, lansoprazole, leminoprazole, pantoprazole, and mixtures thereof, are known to be useful for inhibiting gastric acid secretion in mammals, e.g., humans, by controlling gastric acid secretion at the final step of the acid secretory pathway. These active pharmaceutical ingredients are acid-labile, creating several problems in formulating such acid-labile compounds into oral pharmaceutical dosage forms because of the acidic environment of the stomach, and generally have poor stability. In particular, they would be rapidly decomposed and change color under moist conditions or in an acidic to neutral aqueous solution.

When these compounds are formulated into pharmaceutical preparations for oral administration, they require special techniques to avoid contact of drug with gastric acid of the stomach. One technique that is commonly used is to coat the acid-labile compound, or its granules or pellets, with an enteric coating, which is insoluble in aqueous acidic conditions and soluble in aqueous neutral to alkaline conditions. However, the materials used in enteric coatings are themselves acidic, which can cause decomposition of the acid-labile compound. Such decomposition occurs even during the enteric coating process, which results in the coloration of the surface of the drug-containing core. In order to avoid such problems, an inert subcoating, which is not acidic, can be placed between the core and enteric coating.

For substances that are labile in acid media, but have better stability in neutral to alkaline media, it can be advantageous to add alkaline inactive excipients in order to increase the stability of the active compound during manufacturing and storage. In particular, substituted benzimidazole derivatives such as omeprazole and esomeprazole are not only unstable in acidic conditions but also are not stable in the neutral solid state. Thus, in order to enhance the storage stability, an alkaline base such as sodium bicarbonate can be added to the formulation, and/or the substituted benzimidazole derivatives can be converted to their alkaline salts, which are usually more stable than the free species.

A pharmaceutically active compound of the formulations and methods of the present disclosure is an optical isomer of lansoprazole. Chemically, it is the (+)-isomer of 2-[3-methyl-4-(2,2,2-trifluoroethoxy)pyrid-2-yl]methylsulfinyl benzimidazole, hereinafter referred to as “dexlansoprazole,” and has structural Formula I.

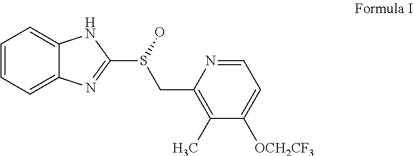

Dexlansoprazole was approved for sale in the U.S.A. on Jan. 30, 2009 and it is presently commercially available in products sold as DEXILANT®, as 30 mg and 60 mg delayed release capsules marketed by Takeda. The inactive excipients of DEXILANT® include sugar spheres, magnesium carbonate, sucrose, low-substituted hydroxypropyl cellulose, titanium dioxide, hydroxypropyl cellulose, hypromellose 2910, talc, methacrylic acid copolymer, polyethylene glycol 8000, triethyl citrate, polysorbate 80, and colloidal silicon dioxide. The capsule shell is made of hypromellose, carrageenan and potassium chloride. Based on the capsule shell color, blue contains FD&C Blue No. 2 and aluminum lake, and gray contains ferric oxide and aluminum lake, and both contain titanium dioxide.

U.S. Pat. Nos. 6,462,058 and 6,664,276 disclose crystalline forms of dexlansoprazole or a salt thereof. U.S. Pat. Nos. 4,628,098, 4,786,505, 4,853,230, 5,689,333, 5,045,321, 5,093,132, and 5,433,959 teach various stabilizing agents for their disclosed benzimidazole derivatives in core tablets. These patents also show that such compounds are stable in the presence of basic inorganic salts of magnesium, calcium, potassium and sodium. The stability is further consolidated by separating acid labile benzimidazoles from the acidic components of the enteric coating by interposing an intermediate coating (a subcoating).

U.S. Pat. No. 6,939,971 teaches a method of treating Zollinger-Ellison syndrome, reflux esophagitis and Helicobacter pylori infection, by administering compositions containing crystalline dexlansoprazole.

U.S. Pat. No. 6,013,281, of which the entire content is incorporated by reference, also discloses that a separating layer is formed in situ by direct application of an acidic enteric material onto an alkaline core containing benzimidazoles. U.S. Patent Application Publication No. 2006/0057195 A1 describes stable solid preparations for medicinal use containing amorphous benzimidazole compounds including dexlansoprazole, which are produced by blending an amorphous benzimidazole compound with a nontoxic base such as a basic inorganic salt.

U.S. Pat. No. 7,790,755 discloses a capsule comprising two portions of tablet, granule, or fine granule compositions comprising an active ingredient, wherein the first portion comprises a pH-dependently soluble release-controlled coating layer that contains polymer(s) which are soluble in the pH range of 6.0 to 7.5 and second portion comprises an enteric coat that releases the active ingredient in the pH range of no less than 5.0 to no more than 6.0. U.S. Patent Application Publication No. 2010/0272798 discloses capsules comprising a tablet, granule, or fine granule and a gel-forming polymer, such that the release of an active ingredient is controlled. It further discloses a composition with controlled release of active in a pH range of 6.0 to 7.5.

U.S. Patent Application Publication No. 2007/0141137 describes a capsule preparation, which comprises a medicine unstable to moisture, is stable in a low moisture state, and has pH-independent disintegration properties. International Application Publication No. WO 2010/117756 discloses an enteric coated formulation comprising dexlansoprazole, wherein the formulation contains any of impurity compounds A, B, C, D, or E, in amounts less than about 2 percent by weight of the label dexlansoprazole content, and containing total dexlansoprazole-related impurities in amounts less than about 5 percent by weight of the label dexlansoprazole content, after storage for three months at 40° C. and 75% relative humidity.

There remains a need for pharmaceutical formulations comprising substituted benzimidazole derivatives, including pharmaceutically acceptable salts thereof, for providing effective plasma concentrations of the active agent for extended durations of time.

### SUMMARY

An aspect of the present disclosure relates to stabilized modified release pharmaceutical formulations for oral administration comprising at least one substituted benzimidazole derivative or a salt thereof, wherein the pharmaceutical formulations comprise:

(a) a first fraction, comprising:
(i) a core containing a substituted benzimidazole derivative or a pharmaceutically acceptable salt thereof, at least one stabilizer, and at least one pharmaceutically acceptable excipient; and
(ii) a pH-dependently soluble release-controlled coating layer surrounding the core, wherein the pH-dependently soluble release-controlled coating layer dissolves in a pH range of about 5-6; and

(b) a second fraction, comprising:
(i) a core containing a substituted benzimidazole derivative or a pharmaceutically acceptable salt thereof, at least one stabilizer, and at least one pharmaceutically acceptable excipient; and
(ii) a release-controlled coating layer surrounding the core, wherein the release-controlled coating layer comprises one or more pH-independent polymers and one or more pH-dependently soluble release-controlled coating polymers.

In embodiments, the present disclosure provides stabilized modified release pharmaceutical formulations as described above, wherein the second fraction further comprises one or more pH-dependently soluble release-controlled coating layers, wherein the pH-dependently soluble release-controlled coating layers dissolve in a pH range of about 5-6.

In embodiments, the present disclosure provides stabilized modified release pharmaceutical formulations as described above, but wherein the second fraction comprises:

(i) a core containing substituted benzimidazole derivative or a pharmaceutically acceptable salt thereof, at least one stabilizer, and at least one pharmaceutically acceptable excipient;

(ii) a release-controlled coating layer surrounding the core; and

(iii) a pH-dependently soluble release controlled coating layer surrounding the release-controlled coating layer.

In embodiments, the present disclosure provides stabilized modified release pharmaceutical formulations as described above, but wherein the second fraction comprises:

(i) a core containing a substituted benzimidazole derivative or a pharmaceutically acceptable salt thereof, at least one stabilizer, and at least one pharmaceutically acceptable excipient;

(ii) a pH-dependently soluble release controlled coating layer surrounding the core; and

(iii) a release-controlled coating layer surrounding the pH-dependently soluble release controlled coating layer.

In embodiments, the present disclosure provides stabilized modified release pharmaceutical formulations as described above, but wherein the second fraction comprises:

(i) a core containing a substituted benzimidazole derivative or a pharmaceutically acceptable salt thereof, at least one stabilizer, and at least one pharmaceutically acceptable excipient;

(ii) an inner pH-dependently soluble release controlled coating layer surrounding the core;

(iii) a release-controlled coating layer surrounding the inner pH-dependently soluble release controlled coating layer; and

(iv) an outer pH-dependently soluble release controlled coating layer surrounding the release-controlled coating layer.

An aspect of the present disclosure relates to stabilized modified release pharmaceutical formulations for oral administration, comprising at least one substituted benzimidazole derivative or a salt thereof, wherein the pharmaceutical formulations comprise:

(a) a first fraction, comprising:
(i) a core containing a substituted benzimidazole derivative or a pharmaceutically acceptable salt thereof, at least one stabilizer, and at least one pharmaceutically acceptable excipient; and
(ii) a pH-dependently soluble release-controlled coating layer surrounding the core, wherein the pH-dependently soluble release-controlled coating layer dissolves in a pH range of about 6-6.5; and

(b) a second fraction, comprising:
(i) a core containing a substituted benzimidazole derivative or a pharmaceutically acceptable salt thereof, at least one stabilizer, and at least one pharmaceutically acceptable excipient; and
(ii) a pH-dependently soluble release-controlled coating layer surrounding the core, wherein the pH-dependently soluble release-controlled coating layer dissolves in a pH range of about 6.5-8.

An aspect of the present disclosure relates to stabilized modified release pharmaceutical formulations for oral administration, comprising at least one substituted benzimidazole derivative or a salt thereof, wherein the pharmaceutical formulations comprise:

(a) an inert core;

(b) a first layer containing a substituted benzimidazole derivative or a pharmaceutically acceptable salt thereof, at least one stabilizer, and at least one pharmaceutically acceptable excipient surrounding the core of (a);

(c) a first pH-dependently soluble release-controlled coating layer surrounding the layer of b), wherein the pH-dependently soluble release-controlled coating layer is soluble in a pH range of about 6-8;

(d) a second layer containing a substituted benzimidazole derivative or a pharmaceutically acceptable salt thereof, at least one stabilizer, and at least one pharmaceutically acceptable excipient, coated over the pH-dependently soluble release-controlled coating layer of (c); and

(e) a second pH-dependently soluble release-controlled coating layer surrounding the layer of (d), wherein the pH-dependently soluble release-controlled coating layer is soluble in a pH range of about 4-6.

An aspect of the present disclosure relates to stabilized modified release formulations comprising at least one of rabeprazole, omeprazole, lansoprazole, leminoprazole, pantoprazole, and salts thereof, as a racemic mixture or a single enantiomer thereof.

In embodiments, the present disclosure provides formulations comprising dexlansoprazole, together with one or more excipients.

In embodiments, the present disclosure provides formulations comprising dexlansoprazole, wherein the dexlansoprazole is in amorphous form.

In embodiments, the present disclosure provides stabilized modified release pharmaceutical formulations comprising two fractions, wherein weight ratios between a first and a second fraction vary from about 10:90 to about 90:10.

In embodiments, the present disclosure provides stabilized modified release pharmaceutical formulations comprising two substituted benzimidazole derivative layers, wherein weight ratios of a substituted benzimidazole derivative or salt thereof between a first and a second drug layer vary from about 1:99 to about 99:1.

In embodiments, the present disclosure provides stabilized modified release pharmaceutical formulations wherein in vitro release of active ingredient from a first fraction precedes the release from a second fraction, above about pH 5.

In aspects of the present disclosure, stabilized modified release pharmaceutical formulations exhibit the following in vitro release profile: no greater than about 10% of dexlansoprazole dissolving within about 120 minutes following immersion in 500 mL of pH 1.2, 0.1 N hydrochloric acid, using USP type 2 apparatus with 75 rpm stirring, then at least about 20% of dexlansoprazole dissolving within about 90 minutes, and at least about 70% of dexlansoprazole dissolving within about 200 minutes, following immersion in 900 mL of pH 7.0 phosphate buffer with 5 mM sodium lauryl sulfate (SLS), using the same apparatus.

In embodiments, the present disclosure provides stabilized modified release pharmaceutical formulations, wherein a pH-dependently soluble release-controlled coating layer comprises one or more pH-dependently soluble release-controlling coating polymers.

In embodiments, a stabilizer is present in amounts about 0.1 to about 10 percent by weight of the total formulation.

In embodiments, stabilized modified release pharmaceutical formulations of the present disclosure comprise a drug dissolution enhancer.

In embodiments, a drug dissolution enhancer is present in amounts about 0.1 to about 5 percent by weight of the total formulation.

In embodiments, a drug stabilizer and/or dissolution enhancer may be present in a subcoating.

In embodiments, the disclosure provides processes for preparing stabilized modified release pharmaceutical formulations, wherein a process comprises:

(a) preparing a first fraction by:
(i) applying a layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying to obtain cores;
(ii) optionally, applying an intermediate coating layer over the cores of step (i); and
(iii) applying a pH-dependently soluble release-controlled coating layer over the core of (i) or the intermediate coating of (ii), wherein the pH-dependently soluble release-controlled coating layer dissolves in a pH range of about 5-6;

(b) preparing a second fraction by:
(i) applying a layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying to obtain cores;
(ii) optionally, applying an intermediate coating layer over the cores of step (i); and
(iii) applying a release-controlled coating layer over the cores of step (i) or the intermediate coating of (ii), wherein the release-controlled coating layer comprises one or more pH-independent polymers and one or more pH-dependently soluble release-controlled coating polymers; and

c) mixing the first fraction particles obtained in step (a) and the second fraction particles obtained in step (b), and filling into capsules or compressing into tablets.

In embodiments, the disclosure provides processes for preparing stabilized modified release pharmaceutical formulations, wherein a process comprises:

(a) preparing a first fraction by:
(i) applying a layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying to obtain cores;
(ii) optionally, applying an intermediate coating layer over the cores of step (i); and
(iii) applying a pH-dependently soluble release-controlled coating layer over the cores of (i) or the intermediate coating of (ii), wherein the pH-dependently soluble release-controlled coating layer dissolves in a pH range of about 6-6.5;

(b) preparing a second fraction by:
(i) applying a layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying to obtain cores;
(ii) optionally, applying an intermediate coating layer over the cores of step (i); and
(iii) applying a pH-dependently soluble release-controlled coating layer over cores of (i) or the intermediate coating of (ii), wherein the pH-dependently soluble release-controlled coating layer dissolves in a pH range of about 6.5-8; and

c) mixing the first fraction particles obtained in step (a) and the second fraction particles obtained in step (b), and filling into capsules or compressing into tablets.

In embodiments, the disclosure provides processes for preparing stabilized modified release pharmaceutical formulations, wherein the process comprises:

(a) applying a first layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying to obtain cores;

(b) optionally, applying an intermediate coating layer over the cores of step (a);

(c) applying a first pH-dependently soluble release-controlled coating layer over cores of (a) or the intermediate coating of (b), wherein the pH-dependently soluble release-controlled coating layer is soluble in a pH range of about 6-8;

(d) optionally, applying an intermediate coating layer over first pH-dependently soluble release-controlled coating layer of step (c);

(e) applying a second layer of a powder, suspension, dispersion, or solution comprising dexlansoprazole or a pharmaceutically acceptable salt thereof, at least one stabilizer, and at least one pharmaceutically acceptable excipient, onto cores of steps (c) or (d), and drying;

(f) optionally, applying an intermediate coating layer over the cores of step (e);

(g) applying a second pH-dependently soluble release-controlled coating layer surrounding the cores of (e) or the intermediate coating of (f), wherein the pH-dependently soluble release-controlled coating layer is soluble in a pH range of about 4-6; and

(h) filling into capsules or compressing into tablets.

The modified release formulations of the present invention are intended to provide effective plasma concentrations of the active agent for extended durations of time.

Additional embodiments of the present disclosure will be apparent from the following description and examples. As can be appreciated from the foregoing and following description, each and every feature described herein, and each and every combination of two or more of such features, is included within the scope of the present disclosure provided that the features included in such a combination are not mutually inconsistent. In addition, any feature or combination of features may be specifically excluded from any embodiment or aspect. Additional aspects and embodiments are set forth in the following description, particularly when considered in conjunction with the examples.

### BRIEF DESCRIPTION OF THE DRAWINGS

FIG. 1 shows comparative X-ray powder diffraction (XRPD) patterns for a formulation prepared according to Example 20, where A represents the formulation and P represents a similarly prepared placebo formulation (i.e., omitting the active ingredient).

FIG. 2 shows comparative XRPD patterns for a formulation prepared according to Example 20, after storage at 40° C. and 75% relative humidity (RH) for 1 month, where A represents the formulation and P represents a similarly prepared placebo formulation.

FIG. 3 shows comparative XRPD patterns for a formulation prepared according to Example 22, where A represents the formulation and P represents a similarly prepared placebo formulation.

FIG. 4 shows comparative XRPD patterns for a formulation prepared according to Example 22, after storage at 40° C. and 75% RH for 2 months, where A represents the formulation and P represents a similarly prepared placebo formulation.

### DETAILED DESCRIPTION

As used herein the term “substituted benzimidazole derivative” includes proton pump inhibitor drug compounds such as rabeprazole, omeprazole, lansoprazole, leminoprazole, pantoprazole, and mixtures thereof, single enantiomers thereof, pharmaceutically acceptable salts, esters, active metabolites, and prodrugs thereof, and polymorphs, solvates, and hydrates thereof. For purposes of brevity, this class of drugs will be represented herein by the compound dexlansoprazole; however, the scope of the disclosure is not limited to this specific drug, as the principles described will apply to other members of the class.

As used herein, the term “dexlansoprazole” includes the drug compound dexlansoprazole, pharmaceutically acceptable salts, esters, and prodrugs thereof, the active metabolites of dexlansoprazole and the prodrugs thereof, and their polymorphs, solvates, and hydrates. For the purpose of brevity, dexlansoprazole is discussed herein as a representative of the substituted benzimidazole class of drugs, although the scope of the disclosure is not intended to be limited to this compound.

The terms “pharmaceutically acceptable salt” as used herein refers to salts that are known to be non-toxic and are commonly used in pharmaceutical practice. Such pharmaceutically acceptable salts include metal salts, salts with organic bases, salts with basic amino acids, etc. Metal salts include, for example, alkali metal salts, such as sodium salt and potassium salts, and alkaline earth metal salts, such as calcium, magnesium and barium salts. Salts with organic bases include, for example, salts with trimethylamine, triethylamine, pyridine, picoline, ethanolamine, diethanolamine, triethanolamine, dicyclohexylamine, N,N-dibenzylethylenediamine, etc. Salts with basic amino acids include, for example, salts with arginine, lysine, etc. Acid addition salts such as hydrochloride salts and the like are also included.

In the present disclosure, dexlansoprazole and its salts can be used in any crystalline form, amorphous form, or combinations thereof.

The term “excipient” or “pharmaceutically acceptable excipient” means a component of a pharmaceutical product that is not an active ingredient, such as a filler, diluent, disintegrant, etc. The excipients that are useful in preparing the pharmaceutical composition are generally safe, non-toxic and neither biologically nor otherwise undesirable, and are acceptable for veterinary use as well as human pharmaceutical use. An “excipient” or “pharmaceutically acceptable excipient” as used in the specification includes both one and more than one such excipient.

The term “acid-labile compound” means a compound, which is not stable in acidic conditions or which undergoes degradation or hydrolysis via acid or proton catalyzed reactions.

The term “optional” or “optionally” means that the subsequently described element, component or circumstance may or may not be present, so that the disclosure includes instances where the element, component, or circumstance occurs and instances where it does not.

The terms “stability” or “stabilized” or “stabilization” as used in the disclosure refers to both physical and chemical stability. The term “physical stability” refers to maintaining the form of dexlansoprazole such as crystalline or amorphous, and the term “chemical stability” refers to maintaining the content of impurities and/or drug degradation products.

The term “stabilizer” as used in the disclosure refers to substances that maintain the physical and/or chemical stability of the drug in the formulation.

In embodiments, the dexlansoprazole used as the active ingredient is in an amorphous form, wherein said form is retained during the manufacturing of the formulation and also during storage for commercially relevant periods.

“Commercially relevant” periods typically are considered to be about 6 months, 1 year, 2 years, etc., including any intermediate times, generally with products packaged in closed containers. For drug products, storage conditions are specified by regulatory authorities, such as 25° C., frequently with excursions permitted to 15-30° C. It is common in the industry to simulate long-term storage, using “accelerated” stability testing conditions, such as 40° C. and 75% relative humidity (RH), etc., for shorter durations.

Like other substituted benzimidazole derivatives, dexlansoprazole is acid-labile, creating several problems in formulating into oral pharmaceutical dosage forms because of the acidic environment of the stomach. It has poor stability and would be rapidly decomposed and colored under moist conditions or in an acidic to neutral aqueous environment. It requires special techniques to minimize or avoid contact of the drug with gastric acid of the stomach. Even though stabilization of substituted benzimidazole derivatives is known, there remains a need for alternate approaches to prepare stable pharmaceutical formulations comprising dexlansoprazole or a pharmaceutically acceptable salt thereof.

Modified release formulations of the present disclosure comprising at least a substituted benzimidazole derivative, such as lansoprazole or a single enantiomer thereof as an active agent, minimize or avoid contact of the active agent with gastric acid of the stomach and are intended to provide effective plasma concentrations of the active agent for extended durations of time.

Embodiments of the present disclosure provide modified release formulations comprising dexlansoprazole as the active agent, together with one or more excipients.

In embodiments, dexlansoprazole formulations of the present disclosure comprise a single fraction of multi-particulates, such as pellets or mini-tablets, filled into a capsule, wherein the particulates comprise one or more drug layers.

In embodiments, modified release formulations of dexlansoprazole according to the present disclosure comprise at least two fractions, wherein at least one fraction comprises the active agent dexlansoprazole and wherein one or more of the fractions is in the form of immediate release, delayed release, extended release, sustained release, pulsatile release, or prolonged release.

In embodiments, modified release formulations of dexlansoprazole according to the present disclosure comprise at least two fractions, wherein one fraction provides an immediate release pulse of dexlansoprazole and the other fraction provides a delayed release of dexlansoprazole.

In embodiments, modified release formulations of dexlansoprazole according to the present disclosure comprise at least two fractions, wherein one fraction provides a sustained release of dexlansoprazole and the other fraction provides a delayed release of dexlansoprazole.

In embodiments, modified release formulations of dexlansoprazole according to the present disclosure comprise at least two fractions, wherein two fractions provide delayed release of dexlansoprazole.

In embodiments, modified release formulations of dexlansoprazole according to the present disclosure comprise at least two fractions, wherein one fraction provides a sustained release of dexlansoprazole and the other fraction provides a delayed release of dexlansoprazole.

In embodiments, modified release formulations of dexlansoprazole comprise at least two fractions, wherein two fractions provide delayed release of dexlansoprazole such that the drug release of one delayed release fraction precedes the other delayed fraction, while releasing a substantial amount of drug before, at the same time, or after a substantial amount of drug is released from another fraction.

In embodiments, modified release formulations of dexlansoprazole comprise at least two fractions, wherein two fractions are in the form of pH-dependently soluble release-controlled coated formulations, intended to provide delayed release of dexlansoprazole, and wherein at least one delayed release fraction provides drug release almost immediately or in an extended manner.

The term ‘modified release’ (MR) according to the present disclosure implies that the drug release can be delayed release (DR), extended release (ER), sustained release (SR), pulsatile release (PSR) or prolonged release (PR), or a combination of immediate release and one or more of delayed release, extended release, sustained release, pulsatile release, and prolonged release.

The term ‘delayed release’ according to the present disclosure implies that the drug is not substantially released in the stomach region of the gastrointestinal tract (GIT); instead, drug release takes place substantially in the upper part of the intestine or a lower part of the intestinal tract.

The term ‘sustained release’ according to the present disclosure implies that the drug is released with varied quantities substantially throughout the GIT in a controlled manner.

The term ‘pulsatile release’ according to the present disclosure implies that the drug is released as one or more pulses in any part of the GIT, immediately or in a delayed manner.

The term ‘extended release’ according to the present disclosure implies that the drug is not substantially released in the stomach region of the GIT; instead, drug release takes place substantially in the upper part of the intestine over an extended duration of time.

The term ‘prolonged release’ according to the present disclosure implies that no drug is released immediately, i.e., the initial drug release starts after a lag time, followed by immediate release of the drug or in a portion of the GIT or varied quantities of drug release throughout the GIT thereafter.

The term ‘pH-dependently soluble release-controlled coating layer’ according to the present disclosure indicates a coating layer which does not dissolve or decompose in the acidic pH of the stomach and dissolves or decomposes in the intestinal pH, and comprises one or more pH-dependently soluble release controlled polymers.

The term ‘release-controlled coating layer’ according to the present disclosure implies a coating layer having a function of delaying or extending the release of active ingredient, and comprises pH-independent polymers such as ‘diffusion-controlled polymers’, or ‘gel-forming polymers’, or ‘eroding or disintegrating polymers’, or ‘matrix-forming polymer’, or ‘extended-release polymer’, or any combination thereof. Optionally, it may further comprise one or more pH-dependently soluble release controlled polymers.

The term ‘diffusion-controlled coating layer’ according to the present disclosure implies a coating layer that itself is not dissolved and which releases an active ingredient through pores that are formed in the layer and comprises diffusion controlled polymers.

Although some coating polymers are considered to dissolve in aqueous media, the term “dissolve” is intended to also apply to polymers that are not actually soluble, but are degraded or decomposed in such media, and thereby permit the release of a drug into the media due to disruptions or removal of the coating.

The term ‘gel-forming polymer coating layer’ according to the present disclosure implies a coating layer that rapidly forms highly viscous gels upon contacting water, and prolongs the retention time in the digestive tract and comprises gel-forming polymers.

The term “dissolution enhancer” as used in the description refers to substances that increase the rate of dissolution of the drug from the carrier.

In embodiments, dexlansoprazole formulations of the present disclosure are in the form of multi-particulates. The ‘multi-particulates’ according to the present disclosure may be in the form of powders, granules, pellets, spheroids, extrudates, mini-tablets, and the like.

In embodiments, dexlansoprazole formulations of the present disclosure are in the form of multi-particulates made into a unit dosage form, such as capsule.

In embodiments, dexlansoprazole formulations of the present disclosure are in the form of pellets or mini-tablets, filled into a capsule.

In embodiments, dexlansoprazole formulations of the present disclosure are in the form of pellets compressed into a tablet.

In embodiments, dexlansoprazole formulations of the present disclosure comprise a single fraction of multi-particulates, such as pellets or mini-tablets, filled into a capsule, wherein the multi-particulate fraction comprises a core containing the drug for extended release, followed by a layer of drug coating for immediate release. These are further coated with a pH-dependently soluble release controlled polymer, wherein the multi-particulates are optionally coated to form a subcoating layer prior to pH-dependently soluble release controlled coating layer.

In embodiments, dexlansoprazole formulations of the present disclosure comprise a single fraction of multi-particulates, such as pellets or mini-tablets, filled into a capsule, wherein the particulates comprise one or more drug layers.

In embodiments, dexlansoprazole formulations of the present disclosure comprise a single fraction of multi-particulates, such as pellets or mini-tablets, filled into a capsule, wherein the multi-particulate fraction comprises a core containing the drug for extended release, having a layer of drug coating for immediate release. These are further coated with a pH-dependently soluble release-controlled coating layer, wherein the multi-particulates are optionally coated to form a subcoating layer prior to a pH-dependently soluble release-controlled coating layer.

In embodiments, dexlansoprazole formulations of the present disclosure comprise a single fraction of multi-particulates, such as pellets or mini-tablets, filled into a capsule, wherein the multi-particulate fraction comprises a core containing a first fraction of the drug, having a pH-dependently soluble release-controlled coating layer that is soluble in a pH range of about 4-8, further coated with a second drug layer. These are further coated with a pH-dependently soluble release-controlled coating layer that is soluble in a pH range of about 3-7. The multi-particulates are optionally coated to form a subcoating layer prior to pH-dependently soluble release-controlled coating layer.

In embodiments, the present disclosure provides dexlansoprazole modified release formulations comprising a single fraction of multi-particulates, comprising at least two drug layers, wherein weight ratios of drug between a first and second drug layer of the multi-particulates vary from about 1:99 to about 99:1.

In embodiments, formulations of dexlansoprazole are in the form of capsules comprising two fractions of pH-dependently soluble release controlled coated pellets exhibiting different release profiles, filled into a capsule, wherein the pellets comprise an inert core onto which the drug layer is formed, optionally, a subcoating, followed by an pH-dependently soluble release controlled coating layer, such that the pH-dependently soluble release controlled coating layers in the two different fractions disintegrate or dissolve in different pH ranges, or the drug release from one fraction precedes the other fraction while releasing substantial amount of drug before, at the same time, or after the same substantial amount of drug is released from the other fraction.

In embodiments, dexlansoprazole formulations of the present disclosure comprise mini-tablets filled into capsules, wherein the mini-tablets comprise a core comprising dexlansoprazole and a stabilizer, optionally coated with a subcoating, and an pH-dependently soluble release controlled coating layer containing at least one pH dependent polymer that dissolves in a pH range of about 4-8 to release the active agent, followed by a layer comprising dexlansoprazole together with one or more excipients, which is coated with another pH-dependently soluble release controlled coating layer containing at least one pH dependent polymer that dissolves in a pH range of about 3-7 to release the active agent.

In embodiments, the present disclosure relates to stabilized modified release pharmaceutical formulations for oral administration, comprising at least one substituted benzimidazole derivative or a salt thereof, wherein the pharmaceutical formulations comprise:

(a) an inert core;

(b) a first layer containing a substituted benzimidazole derivative or a pharmaceutically acceptable salt thereof, at least one stabilizer, and at least one pharmaceutically acceptable excipient, surrounding the core of (a);

(c) a first pH-dependently soluble release-controlled coating layer surrounding the layer of b), wherein the pH-dependently soluble release-controlled coating layer is soluble in a pH range of about 4-8;

(d) a second layer containing a substituted benzimidazole derivative or a pharmaceutically acceptable salt thereof, at least one stabilizer, and at least one pharmaceutically acceptable excipient, coated over the pH-dependently soluble release-controlled coating layer of (c); and

(e) a second pH-dependently soluble release-controlled coating layer surrounding the layer of (d), wherein the pH-dependently soluble release-controlled coating layer is soluble in a pH range of about 3-7.

In embodiments, the present disclosure relates to stabilized modified release pharmaceutical formulations for oral administration, comprising at least one substituted benzimidazole derivative or a salt thereof, wherein the formulation comprises:

(a) an inert core;

(b) a first layer containing a substituted benzimidazole derivative or a pharmaceutically acceptable salt thereof, at least one stabilizer, and at least one pharmaceutically acceptable excipient, surrounding the core of (a);

(c) a first pH-dependently soluble release-controlled coating layer surrounding the layer of b), wherein the pH-dependently soluble release-controlled coating layer is soluble in a pH range of about 6-8;

(d) a second layer containing substituted benzimidazole derivative or a pharmaceutically acceptable salt thereof, at least one stabilizer, and at least one pharmaceutically acceptable excipient, coated over the pH-dependently soluble release-controlled coating layer of (c); and (e) a second pH-dependently soluble release-controlled coating layer surrounding the layer of (d), wherein the pH-dependently soluble release-controlled coating layer is soluble in a pH range of about 4-6.

In embodiments, the present disclosure relates to stabilized modified release pharmaceutical formulations, comprising:

(a) an inert core;

(b) a first layer containing dexlansoprazole or a pharmaceutically acceptable salt thereof, at least one stabilizer, and at least one pharmaceutically acceptable excipient, surrounding the core of (a);

(c) a first pH-dependently soluble release-controlled coating layer surrounding the layer of b), wherein the pH-dependently soluble release-controlled coating layer is soluble in a pH range of about 6-8;

(d) a second layer containing dexlansoprazole or a pharmaceutically acceptable salt thereof, at least one stabilizer, and at least one pharmaceutically acceptable excipient, coated over the pH-dependently soluble release-controlled coating layer of (c); and

(e) a second pH-dependently soluble release-controlled coating layer surrounding the layer of (d), wherein the pH-dependently soluble release-controlled coating layer is soluble in a pH range of about 4-6.

In embodiments, the present disclosure provides stabilized modified release pharmaceutical formulations comprising two substituted benzimidazole derivative layers, wherein weight ratios of substituted benzimidazole derivative or salt thereof between a first and a second drug layer vary from about 1:99 to about 99:1.

In embodiments, the present disclosure provides stabilized modified release pharmaceutical formulations, wherein a pH-dependently soluble release-controlled coating layer comprises one or more pH-dependently soluble release-controlling coating polymers.

In embodiments, the present disclosure provides dexlansoprazole modified release formulations comprising at least two fractions of multi-particulates, wherein one or more of the fractions are in the form of immediate release, delayed release, extended release, sustained release, pulsatile release, or prolonged release.

In embodiments, modified release formulations of dexlansoprazole according to the present disclosure comprise at least two fractions, wherein one fraction provides a sustained release of dexlansoprazole and another fraction provides a delayed release of dexlansoprazole.

In embodiments, dexlansoprazole formulations of the present disclosure comprise at least two fractions of multi-particulates, such as mini-tablets or pellets, filled into a capsule, wherein one fraction of multi-particulates is coated with a pH-dependently soluble release-controlled coating layer that dissolves in a pH range of about 3-7 to release the active agent, and wherein another fraction of multi-particulates is coated with a pH-dependently soluble release controlled polymer that dissolves in a pH range of about 4-8 to release the active agent, and wherein the multi-particulates in each of the two fractions are optionally coated to form a subcoating layer prior to applying a pH-dependently soluble release-controlling coating layer.

In embodiments, stabilized modified release pharmaceutical formulations according to the present disclosure comprise at least one substituted benzimidazole derivative or a salt thereof, wherein the formulations comprise:

(a) a first fraction, comprising:
(i) a core containing a substituted benzimidazole derivative or a pharmaceutically acceptable salt thereof, at least one stabilizer, and at least one pharmaceutically acceptable excipient; and
(ii) a pH-dependently soluble release-controlled coating layer surrounding the core, wherein the pH-dependently soluble release-controlled coating layer dissolves in a pH range of about 5-6; and

(b) a second fraction, comprising:
(i) a core containing a substituted benzimidazole derivative or a pharmaceutically acceptable salt thereof, at least one stabilizer, and at least one pharmaceutically acceptable excipient; and
(ii) a release-controlled coating layer surrounding the core, wherein the release-controlled coating layer comprises one or more pH-independent polymers and one or more pH-dependently soluble release-controlled coating polymers.

In embodiments, the present disclosure provides stabilized modified release pharmaceutical formulations, wherein a fraction of particles comprises one or more pH-dependently soluble release-controlling coating layers, wherein the pH-dependently soluble release-controlled coating layers dissolves in a pH range of about 5-6.

In embodiments, the present disclosure provides stabilized modified release pharmaceutical formulations, wherein a fraction of particles comprises:

(i) a core containing substituted benzimidazole derivative or a pharmaceutically acceptable salt thereof, at least one stabilizer, and at least one pharmaceutically acceptable excipient;

(ii) a release-controlled coating layer surrounding the core; and

(iii) a pH-dependently soluble release controlled coating layer surrounding the release-controlled coating layer.

In embodiments, the present disclosure provides stabilized modified release pharmaceutical formulations, wherein a fraction of particles, comprises:

(i) a core containing substituted benzimidazole derivative or a pharmaceutically acceptable salt thereof, at least one stabilizer, and at least one pharmaceutically acceptable excipient;

(ii) a pH-dependently soluble release controlled coating layer surrounding the core; and

(iii) a release-controlled coating layer surrounding the pH-dependently soluble release controlled coating layer.

In embodiments, the present disclosure provides stabilized modified release pharmaceutical formulations, wherein a fraction of particles comprises:

(i) a core containing a substituted benzimidazole derivative or a pharmaceutically acceptable salt thereof, at least one stabilizer, and at least one pharmaceutically acceptable excipient;

(ii) an inner pH-dependently soluble release controlled coating layer surrounding the core;

(iii) a release-controlled coating layer surrounding the inner pH-dependently soluble release controlled coating layer; and

(iv) an outer pH-dependently soluble release controlled coating layer surrounding the release-controlled coating layer.

In embodiments, the present disclosure provides stabilized modified release pharmaceutical formulation comprising two fractions of particles, wherein weight ratios between the first and second fraction vary from about 10:90 to about 90:10.

In embodiments, the present disclosure provides stabilized modified release pharmaceutical formulations, wherein a pH-dependently soluble release-controlled coating layer comprises one or more pH-dependently soluble release-controlling coating polymers.

In embodiments, the present disclosure provides stabilized modified release pharmaceutical formulations, wherein a release-controlled coating layer comprises one or more pH-independent polymers.

In embodiments, the present disclosure provides stabilized modified release pharmaceutical formulations, wherein a release-controlled coating layer comprises one or more pH-dependently soluble release-controlled coating polymers.

In embodiments, the present disclosure provides stabilized modified release pharmaceutical formulations, wherein in vitro release of active ingredient from a first fraction precedes the release from a second fraction above about pH 5.

In embodiments, the present application includes stabilized modified release formulations comprising dexlansoprazole or pharmaceutically acceptable salts thereof, which release less than about 10% of contained active ingredient, within about the first 120 minutes after immersion into 750 or 1000 mL of 0.1 N hydrochloric acid (pH 1.2) dissolution medium, using test method 711 “Dissolution” in United States Pharmacopeia 29, United States Pharmacopeial Convention, Inc., Rockville, Md., 2005 (“USP”) and Type 2 apparatus. Substantial release of the drug occurs thereafter, upon immersion into 900 mL of pH 7 phosphate buffer with 5 mM sodium lauryl sulphate, at least about 20% of drug dissolving within about 90 minutes, and at least about 70% of the drug dissolving within about 200 minutes.

Dexlansoprazole is chemically characterized by a benzimidazole moiety. The drug is unstable under oxidative stress conditions and degrades to form a sulphone. Dexlansoprazole is a white to nearly white crystalline powder which melts with decomposition at 140° C. to form the impurities 2-mercapto-1H-benzimidazole and an adduct. The drug is stable when exposed to light. Dexlansoprazole is unstable in acidic conditions as compared to neutral to alkaline conditions. The drug degrades to form a sulphide impurity under acidic conditions. Hence, dexlansoprazole is considered to be a generally unstable molecule.

Among the various degradants and impurities, eight compounds have been identified and are described below.

### 1) Impurity A (Carboxylic Acid), chemically 1-(1H-benzoimidazol-2-yl)-3-methyl-4-oxo-1,4-dihydro-pyridine-2-carboxylic acid, is represented by structural Formula II

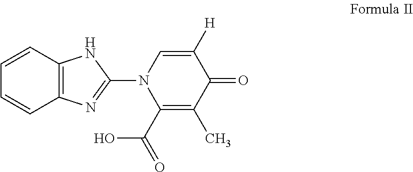

### 2) Impurity B (Hydroxy Benzimidazole), chemically 2-hydroxy benzimidazole, is represented by structural Formula III

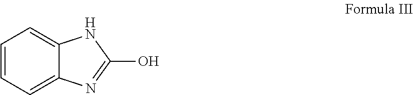

### 3) Impurity C (2-Mercapto benzimidazole), chemically 2-Mercapt-1H-benzimidazole, is represented by structural Formula IV

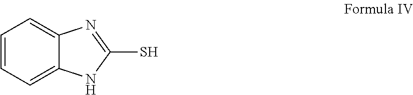

### 4) Impurity D (Sulphone), chemically (2-(((3-methyl-4-(2,2,2-trifluoroethoxy)-2-pyridyl)methyl-4-sulfonyl)benzimidazole), is represented by structural Formula V

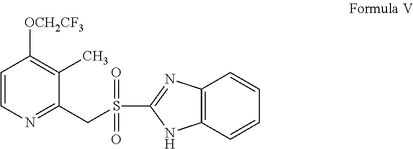

### 5) Impurity E (Sulphide), chemically 3,2-(((3-methyl-4-(2,2,2-trifluoroethoxy)-pyridin-2-yl)methyl)sulfanyl)-1H-benzimadazole, is represented by structural Formula VI

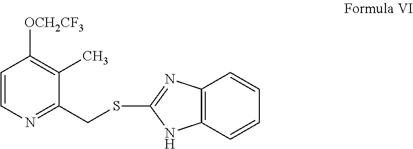

### 6) Impurity F (Cyclised dessulphur—mercapto benzimidazole adduct), chemically 1-(1H-benzoimidazol-2-ylsulfanyl)-1-methyl-2-(2,2,2-trifluoroethoxy)-4-a,5,9b-triaza-indeno[2,1-a]indene, is represented by structural Formula VII

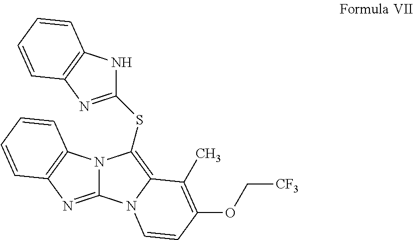

### 7) Impurity G (Cyclized dessulphur-des trifluoroethyl sulphide adduct), chemically 2-{(1H-Benzoimidazol-2-ylsulfanyl)-[1-methyl-2-(2,2,2-trifluoro-ethoxy)-4-a,5,9b-triaza-indeno[2,1-a]inden-10-yl]-methyl}-3-methyl-pyridin-4-ol, is represented by structural Formula VIII.

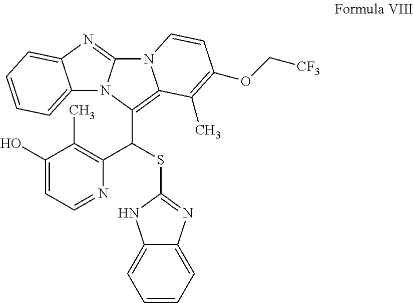

### 8) Impurity H (Cyclized dessulphur adduct), chemically 10-{(1H-benzoimidazol-2-ylsulfanyl)-[3-methyl-4-(2,2,2-trifluoroethoxy)-pyridin-2-yl)-methyl}-1-methyl-2-(2,2,2-trifluoroethoxy)-4-a,5,9b-triaza-indeno[2,1-a]indene, is represented by structural Formula IX.

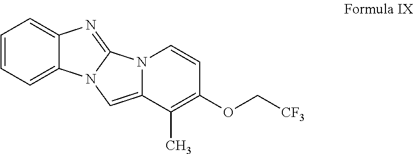

In embodiments, the present application provides stable formulations of dexlansoprazole, which are substantially free of individual degradation impurities.

The term “substantially free” means presence of one or more degradation impurities in amounts less than about 5%, or about 4%, or about 3%, or about 2%, or about 1%, of the label content of dexlansoprazole.

In embodiments, the application relates to stabilized compositions and/or formulations wherein levels of impurity A are less than about 2% of the label content of dexlansoprazole.

In embodiments, the application relates to stabilized compositions and/or formulations wherein levels of impurity B are less than about 2% of the label content of dexlansoprazole.

In embodiments, the application relates to stabilized compositions and/or formulations wherein levels of impurity C are less than about 2% of the label content of dexlansoprazole.

In embodiments, the application relates to stabilized compositions and/or formulations wherein levels of impurity D are less than about 2% of the label content of dexlansoprazole.

In embodiments, the application relates to stabilized compositions and/or formulations wherein levels of impurity E are less than about 2% of the label content of dexlansoprazole.

In embodiments, the application relates to stabilized compositions and/or formulations wherein levels of impurity F are less than about 2% of the label content of dexlansoprazole.

In embodiments, the application relates to stabilized compositions and/or formulations wherein levels of impurity G are less than about 2% of the label content of dexlansoprazole.

In embodiments, the application relates to stabilized compositions and/or formulations wherein levels of impurity H are less than about 2% of the label content of dexlansoprazole.

In embodiments, formulations of the present application may contain any one or more of impurities A, B, C, D, E, F, G, H, or any other drug-related impurities.

In embodiments, the application relates to stable compositions and/or formulations wherein total drug-related impurities, including any combinations of impurities A through H, are less than about 5% of the label content of dexlansoprazole.

In embodiments, the present disclosure relates to stabilized modified release pharmaceutical formulation, wherein a stabilizer present in a drug layer is a water-soluble excipient.

In embodiments, a drug stabilizer may be present in a subcoating.

In embodiments, a stabilizer is present in amounts about 0.1 to about 10 percent by weight of the total formulation.

In embodiments, a substituted benzimidazole derivative or a salt thereof present in first and second fractions is the same or different.

In aspects, stabilized modified release pharmaceutical formulations according to the present disclosure are in the form of granules, pellets, spherules, micro-tablets, mini-tablets, a tablet, a capsule, or a capsule filled with multi-particulates.

In embodiments, stabilized modified release pharmaceutical formulations of the present disclosure are in the form of capsules filled with multi-particulates, wherein multi-particulates of a first fraction comprise:

(a) an inert core;

(b) optionally, a seal coat layer surrounding the core of (a);

(c) a layer of dexlansoprazole or a pharmaceutically acceptable salt thereof, at least one stabilizer, and at least one pharmaceutically acceptable excipient;

(d) optionally, an intermediate coating layer surrounding the drug layer of (c); and

(e) a pH-dependently soluble release-controlled coating layer surrounding the core of (c) or intermediate layer of (d), wherein the pH-dependently soluble release-controlled coating layer dissolves in a pH range of about 5-6.

In embodiments, stabilized modified release pharmaceutical formulations of the present disclosure are in the form of capsules filled with multi-particulates, wherein multi-particulates of a second fraction comprises:

(a) an inert core;

(b) optionally, a seal coat layer surrounding the core of (a);

(c) a layer of dexlansoprazole or a pharmaceutically acceptable salt thereof, at least one stabilizer, and at least one pharmaceutically acceptable excipient;

(d) optionally, an intermediate coating layer surrounding the drug layer of (c);

(e) a pH-dependently soluble release-controlled coating layer surrounding the drug layer of (c) or intermediate layer of (d), wherein the pH-dependently soluble release-controlled coating layers dissolves in a pH range of about 5-6;

(f) a release-controlled coating layer surrounding the pH-dependently soluble release-controlled layer of (e), wherein the release-controlled coating layer comprises one or more pH-independent polymers and one or more pH-dependently soluble release-controlled coating polymers; and

(g) a pH-dependently soluble release-controlled coating layer surrounding the release-controlled coating layer of (f), wherein the pH-dependently soluble release-controlled coating layers dissolves in a pH range of about 5-6.

In embodiments, stabilized modified release pharmaceutical formulations of the present disclosure are in the form of capsules filled with multi-particulates, wherein multi-particulates of a second fraction comprise:

(a) an inert core;

(b) optionally, a seal coat layer surrounding the core;

(c) a layer of dexlansoprazole or a pharmaceutically acceptable salt thereof, at least one stabilizer, and at least one pharmaceutically acceptable excipient;

(d) optionally, an intermediate coating layer surrounding the drug layer of (c);

(e) a release-controlled coating layer surrounding the drug layer of (c) or the coating layer of (d), wherein the release-controlled coating layer comprises one or more pH-independent polymers and one or more pH-dependently soluble release-controlled coating polymers; and

(f) a pH-dependently soluble release-controlled coating layer surrounding the release-controlled coating layer, wherein the pH-dependently soluble release-controlled coating layers dissolves in a pH range of about 5-6.

In embodiments, stabilized modified release pharmaceutical formulations according to the present disclosure are in the form of a capsule filled with multi-particulates, wherein multi-particulates of a second fraction comprise:

(a) an inert core;

(b) optionally, a seal coat layer surrounding the core;

(c) a layer of dexlansoprazole or a pharmaceutically acceptable salt thereof, at least one stabilizer, and at least one pharmaceutically acceptable excipient;

(d) optionally, an intermediate coating layer surrounding the drug layer of (c);

(e) a pH-dependently soluble release-controlled coating layer surrounding the drug layer of (c) or intermediate layer of d), wherein the pH-dependently soluble release-controlled coating layer dissolves in a pH range of about 5-6; and

(f) a release-controlled coating layer over (e), wherein the release-controlled coating layer comprises one or more pH-independent polymers and one or more pH-dependently soluble release-controlled coating polymers.

In embodiments, modified release pharmaceutical formulations of the present disclosure are in the form of capsules filled with multi-particulates, wherein a particle comprises:

(a) a core, comprising dexlansoprazole or a pharmaceutically acceptable salt thereof, at least one stabilizer, and at least one pharmaceutically acceptable excipient;

(b) optionally, an intermediate coating layer surrounding the core;

(c) a pH-dependently soluble release-controlled coating layer surrounding the core of (a) or intermediate layer of (b), wherein the pH-dependently soluble release-controlled coating layer is soluble in a pH range of about 4-8;

(d) optionally, an intermediate coating layer surrounding the pH-dependently soluble release-controlled coating layer of (c);

(e) a drug layer coated over the pH-dependently soluble release-controlled coating layer of (c) or intermediate layer of (d);

(f) optionally, an intermediate coating layer surrounding the drug layer of (e); and

(g) a pH-dependently soluble release-controlled coating layer surrounding the drug layer of (e) or intermediate layer of (d), wherein the pH-dependently soluble release-controlled coating layer is soluble in a pH range of about 3-7.

In embodiments, modified release pharmaceutical formulations of the present disclosure are in the form of capsules filled with multi-particulates, wherein a particle comprises:

(a) a core, comprising dexlansoprazole or a pharmaceutically acceptable salt thereof, at least one stabilizer, and at least one pharmaceutically acceptable excipient;

(b) optionally, an intermediate coating layer surrounding the core;

(c) a pH-dependently soluble release-controlled coating layer surrounding the core of (a) or intermediate layer of (b), wherein the pH-dependently soluble release-controlled coating layer is soluble in a pH range of about 3-7;

(d) a drug layer coated over the pH-dependently soluble release-controlled coating layer of (c); and

(e) a film coating layer surrounding the drug layer of (d).

In embodiments, stable modified release pharmaceutical formulations of the present disclosure are in the form of capsules filled with multi-particulates, wherein a particle comprises:

(a) a core, comprising dexlansoprazole or a pharmaceutically acceptable salt thereof, a diffusion-controlled release polymer, at least one stabilizer, and at least one pharmaceutically acceptable excipient;

(b) a drug layer coated over the core;

(c) optionally, an intermediate coating layer surrounding the drug layer of (b); and

(d) a pH-dependently soluble release-controlled coating layer surrounding the drug layer of (b) or intermediate layer of (c), wherein the pH-dependently soluble release-controlled coating layer is soluble in a pH range of about 3-8.

In embodiments, the present disclosure provides stabilized dexlansoprazole modified release pharmaceutical formulations comprising two dexlansoprazole-containing layers, wherein weight ratios of dexlansoprazole between first and second drug layers vary from about 1:99 to about 99:1.

In embodiments, the present disclosure provides stabilized dexlansoprazole modified release pharmaceutical formulations comprising two fractions, wherein weight ratios between the first and second fractions vary from about 10:90 to about 90:10.

In embodiments, the present disclosure provides stabilized modified release pharmaceutical formulations, wherein a pH-dependently soluble release-controlled coating layer comprises one or more pH-dependently soluble release-controlling coating polymers.

In embodiments, the present disclosure provides stabilized modified release pharmaceutical formulations, wherein a release-controlled coating layer comprises one or more pH-independent polymers.

In embodiments, the present disclosure provides stabilized modified release pharmaceutical formulations, wherein a release-controlled coating layer comprises one or more pH-dependently soluble release-controlling coating polymers.

In embodiments, dexlansoprazole formulations of the present disclosure comprise at least two fractions of multi-particulates, such as mini-tablets or pellets, filled into a capsule, wherein one fraction of multi-particulates is coated with a pH-dependently soluble release controlling polymer that dissolves or decomposes in a pH range of about 4-7 to release the active agent in the upper part of the small intestine, and wherein another fraction of multi-particulates is coated with a release-controlling coating layer, followed by a pH-dependently soluble release controlling polymer that dissolves or decomposes in a pH range of about 4-7 to release the active agent in lower part of small intestine or in large intestine, and wherein the multi-particulates in each of the two fractions are optionally coated to form a subcoating layer.

In embodiments, dexlansoprazole formulations of the present disclosure comprise at least two fractions of multi-particulates, such as mini-tablets or pellets, filled into a capsule, wherein one fraction of multi-particulates is coated with a pH-dependently soluble release controlling coating layer that dissolves in a pH range of about 4-7 to release the active agent in the upper part of small intestine, and wherein another fraction of multi-particulates is coated with a first pH-dependently soluble release controlling coating layer, followed by a release-controlled coating layer, and finally by a second pH-dependently soluble release controlling coating layer that dissolves in a pH range of about 4-7 to release the active agent in a lower part of the small intestine or in the large intestine, and wherein the multi-particulates in each of the two fractions are optionally coated to form a subcoating layer.

In embodiments, formulations of dexlansoprazole are in the form of capsules comprising two fractions of coated mini-tablets (a) and (b), wherein one of the fractions forms about 10-95% by weight of the total weight of mini-tablets filled into a capsule, and wherein: (a) one fraction of mini-tablets comprises dexlansoprazole, at least one stabilizer in a core, and at least one pH-dependently soluble release-controlled coating polymer in a coating; and (b) the other fraction of mini-tablets comprises dexlansoprazole, at least one stabilizer, and at least one matrix forming polymer in a core, and at least one pH-dependently soluble release-controlled coating polymer in a coating.

In embodiments, formulations of dexlansoprazole are in the form of capsules comprising two fractions of coated mini-tablets (a) and (b), wherein one of the fractions forms about 10-95% by weight of the total weight of mini-tablets filled into a capsule, and wherein: (a) one fraction of mini-tablets comprises dexlansoprazole, at least one stabilizer in a core, and at least one release-controlled polymer in a coating; and (b) the other fraction of mini-tablets comprises dexlansoprazole and at least one stabilizer in a core, and at least one pH-dependently soluble release-controlled coating polymer in a coating.

In embodiments, formulations of dexlansoprazole are in the form of capsules comprising two fractions of coated mini-tablets (a) and (b), wherein one of the fractions forms about 10-95% by weight of the total weight of mini-tablets filled into a capsule, and wherein: (a) one fraction of minitablets comprises an inert core, a dexlansoprazole-containing layer over the core, optionally, a subcoating, and at least one coating comprising at least one release-controlled polymer; and (b) the other fraction of mini-tablets comprises an inert core, a dexlansoprazole-containing layer over the core, optionally, a subcoating, and at least one coating comprising at least one pH-dependently soluble release-controlled coating polymer.

In embodiments, formulations of dexlansoprazole are in the form of capsules comprising two fractions of coated mini-tablets (a) and (b), wherein one of the fractions forms about 5-98% by weight of the total weight of mini-tablets filled into a capsule, and wherein: (a) one fraction of mini-tablets comprises dexlansoprazole, at least one stabilizer in a core, optionally, a subcoating, and at least one coating comprising at least one pH-dependently soluble release-controlled coating layer that dissolves in a pH range of about 4-7 to release the active agent; and (b) the other fraction of mini-tablets comprises dexlansoprazole and at least one stabilizer in a core, optionally a subcoating, and at least one coating comprising at least one pH-dependently soluble release-controlled coating layer which dissolves in a pH range of about 4-7 to release the active agent, together with at least one pore former.

In embodiments, formulations of dexlansoprazole are in the form of capsules comprising two fractions of coated pellets (a) and (b), wherein one of the fractions forms about 5-98% by weight of the total weight of pellets filled into a capsule, and wherein: (a) one fraction of pellets comprises a core comprising dexlansoprazole or a pharmaceutically acceptable salt thereof, at least one stabilizer and at least one pharmaceutically acceptable excipient, optionally, an intermediate coating surrounding the core, and a pH-dependently soluble release-controlled coating layer that dissolves in a pH range of about 5-6 to release the active agent; and (b) the other fraction of pellets comprises a core, comprising dexlansoprazole or a pharmaceutically acceptable salt thereof, at least one stabilizer and at least one pharmaceutically acceptable excipient, a release-controlled coating layer surrounding the core, wherein the release-controlled coating layer is a diffusion-controlled coating layer; and a pH-dependently soluble release-controlled coating layer that dissolves in a pH range of about 5-6 to release the active agent; wherein the fraction (b) does not completely release the active agent at pH 5-6, instead about 10% to about 100% of the active agent in fraction (b) is released at pH values about 5-7.

In embodiments, formulations of dexlansoprazole are in the form of capsules comprising two fractions of coated pellets (a) and (b), wherein one of the fractions forms about 5-98% by weight of the total weight of pellets filled into a capsule, and wherein: (a) one fraction of pellets comprises a core comprising dexlansoprazole or a pharmaceutically acceptable salt thereof, at least one stabilizer, and at least one pharmaceutically acceptable excipient, optionally, an intermediate coating surrounding the core, and a pH-dependently soluble release-controlled coating layer that dissolves in a pH range of about 5-6 to release the active agent; and (b) the other fraction of pellets comprises a core, comprising dexlansoprazole or a pharmaceutically acceptable salt thereof, at least one stabilizer, and at least one pharmaceutically acceptable excipient, optionally, an intermediate coating surrounding the core, a release-controlled coating layer, wherein the release-controlled coating layer comprises diffusion-controlled polymer, gel-forming polymer, matrix-forming polymer, extended-release polymer, or any combination thereof, and a pH-dependently soluble release-controlled coating layer that dissolves in a pH range of about 5-6 to release the active agent; and wherein the fraction (b) does not completely release the active agent at pH 5-6, instead about 10% to about 100% of the active agent in fraction (b) is released at pH values about 5-7.

In embodiments, formulations of dexlansoprazole are in the form of capsules comprising two fractions of coated pellets (a) and (b), wherein one of the fractions forms about 5-98% by weight of the total weight of pellets filled into a capsule, and wherein: (a) one fraction of pellets comprises a core comprising dexlansoprazole or a pharmaceutically acceptable salt thereof, at least one stabilizer and at least one pharmaceutically acceptable excipient, optionally, an intermediate coating surrounding the core, and a pH-dependently soluble release-controlled coating layer that dissolves in a pH range of about 5-6 to release the active agent; and (b) the other fraction of pellets comprises a core comprising dexlansoprazole or a pharmaceutically acceptable salt thereof, at least one stabilizer and at least one pharmaceutically acceptable excipient, optionally, an intermediate coating surrounding the core, a pH-dependently soluble release-controlled coating layer that dissolves in a pH range of about 5-6 to release the active agent, and a release-controlled coating layer, wherein the release-controlled coating layer comprises a diffusion-controlled polymer, gel-forming polymer, matrix-forming polymer, extended-release polymer, or any combination thereof, followed by an external pH-dependently soluble release-controlled coating layer that dissolves in a pH range of about 5-6; and wherein the fraction (b) does not completely release the active agent at pH 5-6, instead about 10% to about 100% of the active agent in fraction (b) is released at pH values about 5-7.

In embodiments, the present disclosure relates to stabilized modified release pharmaceutical formulations for oral administration comprising at least one substituted benzimidazole derivative or a salt thereof, wherein the pharmaceutical formulations comprise: (a) a first fraction, comprising: (i) a core containing a substituted benzimidazole derivative or a pharmaceutically acceptable salt thereof, at least one stabilizer, and at least one pharmaceutically acceptable excipient; and

(ii) a pH-dependently soluble release-controlled coating layer surrounding the core, wherein the pH-dependently soluble release-controlled coating layer dissolves in a pH range of about 6-6.5; (b) a second fraction, comprising:(i) a core containing substituted benzimidazole derivative or a pharmaceutically acceptable salt thereof, at least one stabilizer, and at least one pharmaceutically acceptable excipient; and
(ii) a pH-dependently soluble release-controlled coating layer surrounding the core, wherein the pH-dependently soluble release-controlled coating layer dissolves in a pH range of about 6.5-8.

In embodiments, formulations of dexlansoprazole are in the form of capsules comprising two fractions of coated pellets (a) and (b), wherein one of the fractions forms about 5-98% by weight of the total weight of pellets filled into a capsule, and wherein: (a) one fraction of pellets comprises a core comprising dexlansoprazole or a pharmaceutically acceptable salt thereof, at least one stabilizer and at least one pharmaceutically acceptable excipient, optionally, an intermediate coating surrounding the core, and a pH-dependently soluble release-controlled coating layer that dissolves in a pH range of about 6-6.5 to release the active agent; and (b) the other fraction of pellets comprises a core, comprising dexlansoprazole or a pharmaceutically acceptable salt thereof, at least one stabilizer and at least one pharmaceutically acceptable excipient, an intermediate coating surrounding the core; and a pH-dependently soluble release-controlled coating layer that dissolves in a pH range of about 6.5-8 to release the active agent; wherein release from fraction (a) precedes release from fraction (b) and fraction (b) does not substantially release the active agent below pH 6.5, instead about 10% to about 100% of the active agent in fraction (b) is released at pH values above about 6.5.

In embodiments, formulations of dexlansoprazole are in the form of capsules comprising two fractions of coated pellets (a) and (b), wherein one of the fractions forms about 5-98% by weight of the total weight of pellets filled into a capsule, and wherein: (a) one fraction of pellets comprises a core comprising dexlansoprazole or a pharmaceutically acceptable salt thereof, at least one stabilizer and at least one pharmaceutically acceptable excipient, optionally, an intermediate coating surrounding the core, a pH-dependently soluble release-controlled coating layer that dissolves in a pH range of about 5-8 to release the active agent, optionally, an intermediate coating surrounding a drug layer, and a pH-dependently soluble release-controlled coating layer that dissolves in a pH range of about 5-6 to release the active agent; and (b) the other fraction of pellets comprises a core comprising dexlansoprazole or a pharmaceutically acceptable salt thereof, at least one stabilizer and at least one pharmaceutically acceptable excipient, optionally, an intermediate coating surrounding the core, a pH-dependently soluble release-controlled coating layer that dissolves in a pH range of about 6-8 to release the active agent; and wherein fraction (a) does not completely release the active agent at pH 5-6, instead about 10% to about 90% of the active agent in fraction (a) is released at pH values about 6-8.

In embodiments, formulations of dexlansoprazole are in the form of capsules comprising two fractions of coated pellets (a) and (b), wherein one of the fractions forms about 5-98% by weight of the total weight of pellets filled into a capsule, and wherein: (a) one fraction of pellets comprises a core comprising dexlansoprazole or a pharmaceutically acceptable salt thereof, at least one stabilizer and at least one pharmaceutically acceptable excipient, optionally, an intermediate coating surrounding the core, a pH-dependently soluble release-controlled coating layer that dissolves in a pH range of about 5-6 to release the active agent, a drug layer, and a film coating layer; and (b) the other fraction of pellets comprises a core comprising dexlansoprazole or a pharmaceutically acceptable salt thereof, at least one stabilizer and at least one pharmaceutically acceptable excipient, optionally, an intermediate coating surrounding the core, a pH-dependently soluble release-controlled coating layer that dissolves in a pH range of about 6-8 to release the active agent; and wherein fraction (a) releases part of the active agent below pH 5, the remaining quantity of the active agent in fraction (a) being released at pH values about 5-6.

In embodiments, formulations of dexlansoprazole are in the form of capsules comprising two fractions of coated pellets (a) and (b), wherein one of the fractions forms about 5-98% by weight of the total weight of pellets filled into a capsule, and wherein: (a) one fraction of pellets comprises a core comprising dexlansoprazole or a pharmaceutically acceptable salt thereof, a diffusion-controlled release polymer, at least one stabilizer and at least one pharmaceutically acceptable excipient, a drug layer, optionally, an intermediate coating surrounding the core, and a pH-dependently soluble release-controlled coating layer that dissolves in a pH range of about 5-8 to release the active agent; and (b) the other fraction of pellets comprises a core comprising dexlansoprazole or a pharmaceutically acceptable salt thereof, at least one stabilizer and at least one pharmaceutically acceptable excipient, optionally, an intermediate coating surrounding the core, and a pH-dependently soluble release-controlled coating layer that dissolves in a pH range of about 6-8 to release the active agent; and wherein fraction (a) does not completely release the active agent at pH 5-6, instead about 10% to about 90% of the active agent in fraction (a) is released at pH values about 5-8.

In embodiments, formulations of dexlansoprazole are in the form of capsules comprising two fractions of coated pellets (a) and (b), wherein one of the fractions forms about 5-98% by weight of the total weight of pellets filled into a capsule, and wherein: (a) one fraction of pellets comprises a core comprising dexlansoprazole or a pharmaceutically acceptable salt thereof, at least one stabilizer and at least one pharmaceutically acceptable excipient, optionally, an intermediate coating surrounding the core, and a pH-dependently soluble release-controlled coating layer that dissolves in a pH range of about 5-8 to release the active agent; and (b) the other fraction of pellets comprises a core comprising dexlansoprazole or a pharmaceutically acceptable salt thereof, at least one stabilizer and at least one pharmaceutically acceptable excipient, optionally, an intermediate coating surrounding the core, and a pH-dependently soluble release-controlled coating layer that dissolves in a pH range of above 6 to about 8 to release the active agent; and wherein fraction (a) does not completely release the active agent at pH 5-6, instead about 10% to about 90% of the active agent in fraction (a) is released at pH values about 6-8.

In embodiments, formulations of dexlansoprazole are in the form of capsules comprising two fractions of coated pellets (a) and (b), wherein one of the fractions forms about 5-98% by weight of the total weight of pellets filled into a capsule, and wherein: (a) one fraction of pellets comprises a core comprising dexlansoprazole or a pharmaceutically acceptable salt thereof, at least one stabilizer and at least one pharmaceutically acceptable excipient, optionally, an intermediate coating surrounding the core, and a pH-dependently soluble release-controlled coating layer that dissolves in a pH range of about 5-8 to release the active agent; and (b) the other fraction of pellets comprises a core comprising dexlansoprazole or a pharmaceutically acceptable salt thereof, at least one stabilizer and at least one pharmaceutically acceptable excipient, optionally, an intermediate coating surrounding the core, a release-controlled coating layer, wherein the release-controlled coating layer comprises a diffusion-controlled polymer, gel-forming polymer, matrix-forming polymer, extended-release polymer, or any combination thereof, and a pH-dependently soluble release-controlled coating layer that dissolves in a pH range of about 5-6 to release the active agent; and wherein fraction (b) does not completely release the active agent at pH 5-6, instead about 10% to about 100% of the active agent in fraction (b) is released at pH values about 5-8.

In embodiments, formulations of dexlansoprazole are in the form of capsules comprising two fractions of coated pellets (a) and (b), wherein one of the fractions forms about 5-98% by weight of the total weight of pellets filled into a capsule, and wherein: (a) one fraction of pellets comprises a core comprising dexlansoprazole or a pharmaceutically acceptable salt thereof, a diffusion-controlled release polymer, at least one stabilizer and at least one pharmaceutically acceptable excipient, optionally, an intermediate coating surrounding the core, and a pH-dependently soluble release-controlled coating layer that dissolves in a pH range of about 5-8 to release the active agent; and (b) the other fraction of pellets comprises a core comprising dexlansoprazole or a pharmaceutically acceptable salt thereof, at least one stabilizer and at least one pharmaceutically acceptable excipient, optionally, an intermediate coating surrounding the core, a pH-dependently soluble release-controlled coating layer that dissolves in a pH range of about 5-6 to release the active agent, and a release-controlled coating layer, wherein the release-controlled coating layer comprises a diffusion-controlled polymer, gel-forming polymer, matrix-forming polymer, extended-release polymer, or any combination thereof; and wherein fraction (b) does not completely release the active agent at pH 5-6, instead about 10% to about 100% of the active agent in fraction (b) is released at pH values about 5-8.

In embodiments, formulations of dexlansoprazole are in the form of tablets comprising two fractions of coated pellets or mini-tablets (a) and (b), wherein one of the fractions forms about 5-98% by weight of the total weight of pellets or mini-tablets compressed into a tablet, and wherein: (a) one fraction of pellets or mini-tablets comprises a core comprising dexlansoprazole or a pharmaceutically acceptable salt thereof, at least one stabilizer and at least one pharmaceutically acceptable excipient, optionally, an intermediate coating surrounding the core, and a pH-dependently soluble release-controlled coating layer that dissolves in a pH range of about 5-6 to release the active agent; and (b) the other fraction of pellets or mini-tablets comprises a core comprising dexlansoprazole or a pharmaceutically acceptable salt thereof, at least one stabilizer and at least one pharmaceutically acceptable excipient, optionally, an intermediate coating surrounding the core, and a pH-dependently soluble release-controlled coating layer that dissolves in a pH range of about 6-8 to release the active agent. The pellets (a) and (b) are blended with pharmaceutically acceptable excipients, followed by compression of the blend into tablets. Optionally, the tablets are coated with a film forming polymer.

In an aspect, this disclosure includes methods of preparing modified release formulations of dexlansoprazole, embodiments comprising:

(a) applying a layer of a powder, suspension, dispersion, or solution comprising dexlansoprazole, at least one stabilizer, and at least one pharmaceutically acceptable excipient, onto pharmacologically inert particles, and drying, if needed, to obtain cores;

(b) optionally, applying an intermediate coating layer over the cores of step (a);

(c) applying a pH-dependently soluble release-controlled coating layer over cores of (a) or the intermediate coating layer of (b); and

(d) combining coated particles with one or more pharmaceutically acceptable excipients.

In embodiments, the disclosure provides processes for preparing stabilized modified release pharmaceutical formulations, wherein a process comprises:

(a) preparing a first fraction by:
(i) applying a layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying, if needed, to obtain cores;
(ii) optionally, applying an intermediate coating layer over the cores of step (i); and
(iii) applying a pH-dependently soluble release-controlled coating layer over the cores of (i) or the intermediate coating of (ii), wherein the pH-dependently soluble release-controlled coating layer dissolves in a pH range of about 5-6;

(b) preparing a second fraction by:
(i) applying a layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying, if needed, to obtain cores;
(ii) optionally, applying an intermediate coating layer over the cores of step (i); and
(iii) applying a release-controlled coating layer over the cores of step (i) or the intermediate coating of (ii), wherein the release-controlled coating layer comprises one or more pH-independent polymers and one or more pH-dependently soluble release-controlled coating polymers; and

c) combining the first fraction particles obtained in step (a) and the second fraction particles obtained in step (b), and filling into capsules or compressing into tablets.

In embodiments, the disclosure provides processes for preparing stabilized modified release pharmaceutical formulations, wherein a process comprises:

(a) preparing a first fraction by:
(i) applying a layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying, if needed, to obtain cores;
(ii) optionally, applying an intermediate coating layer over the cores of step (i); and
(iii) applying a pH-dependently soluble release-controlled coating layer over the cores of (i) or the intermediate coating of (ii), wherein the pH-dependently soluble release-controlled coating layer dissolves in a pH range of about 6-6.5;

(b) preparing a second fraction by:
(i) applying a layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying, if needed, to obtain cores;
(ii) optionally, applying an intermediate coating layer over the cores of step (i); and
(iii) applying a pH-dependently soluble release-controlled coating layer over cores of (i) or the intermediate coating of (ii), wherein the pH-dependently soluble release-controlled coating layer dissolves in a pH range of about 6.5-8; and

c) combining the first fraction particles obtained in step (a) and the second fraction particles obtained in step (b), and filling into capsules or compressing into tablets.

In embodiments, the disclosure provides processes for preparing stabilized modified release pharmaceutical formulations, wherein a process comprises:

(a) applying a first layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying, if needed, to obtain cores;

(b) optionally, applying an intermediate coating layer over the cores of step (a);

(c) applying a first pH-dependently soluble release-controlled coating layer over cores of (a) or the intermediate coating of (b), wherein the pH-dependently soluble release-controlled coating layer is soluble in a pH range of about 6-8;

(d) optionally, applying an intermediate coating layer over first pH-dependently soluble release-controlled coating layer of step (c);

(e) applying a second layer of a powder, suspension, dispersion, or solution comprising dexlansoprazole or a pharmaceutically acceptable salt thereof, at least one stabilizer, and at least one pharmaceutically acceptable excipient, onto cores of steps (c) or (d), and drying, if needed;

(f) optionally, applying an intermediate coating layer over the cores of step (e);

(g) applying a second pH-dependently soluble release-controlled coating layer surrounding the cores of (e) or the intermediate coating of (f), wherein the pH-dependently soluble release-controlled coating layer is soluble in a pH range of about 4-6; and

(h) filling into capsules or compressing into tablets.

In an aspect, this disclosure includes stabilized pharmaceutical formulations of dexlansoprazole which may be prepared by spray drying a suspension or solution of dexlansoprazole and a water soluble sugar derivative, with or without an organic base, optionally together with one or more pharmaceutically acceptable excipients. Alternatively, dexlansoprazole formulations may also be prepared using fluid bed granulation techniques, where a solution of dexlansoprazole, with or without a stabilizer, optionally together with one or more pharmaceutically acceptable excipients, is sprayed onto inert cores or layered onto inert cores.

In specific embodiments, a formulation of the present disclosure may be prepared by a process including: (a) dissolving dexlansoprazole or a pharmaceutically acceptable salt thereof in an organic solvent; (b) optionally, adding one or more pharmaceutically acceptable excipients such as a stabilizer, a binder, polymer, and/or a disintegrant to the solution; (c) spraying the solution onto a substrate comprising at least one diluent, optionally together with a disintegrant, to obtain a granulated mass; (d) drying the granulated mass; (e) optionally, milling the mass; (f) mixing one or more excipients such as a diluent, disintegrant, lubricant, and/or glidant with the dried mass of (d) or milled mass of (e); (g) compressing the material of (f) to form mini-tablets; (h) optionally subcoating the mini-tablets; (i) coating the mini-tablets of (g) or (h) with a pH dependent polymer or a release-controlled polymer; and (j) filling coated mini-tablets into capsules. In embodiments, a capsule comprises at least two fractions of coated mini-tablets, wherein one fraction is coated with one or more release-controlling polymers and the other fraction is coated with one or more pH dependent polymers. In embodiments, a capsule comprises at least two fractions of coated mini-tablets, wherein both fractions are coated with one or more release-controlled polymer, or one or more pH dependent polymers.

In embodiments, dexlansoprazole formulations may be prepared using powder layering techniques, wherein a drug layering powder comprising dexlansoprazole, with or without a stabilizer, and optionally together with one or more pharmaceutically acceptable excipients, is layered onto inert cores while being sprayed with a binder solution. In a specific embodiment, a formulation of the present disclosure may be prepared by a process including:

(a) preparing a drug layering powder by mixing a diluent, optionally together with one or more pharmaceutically acceptable excipients such as a stabilizer and/or a disintegrant;

(b) preparing a binder solution;

(c) coating sugar spheres with drug layering powder, while being sprayed with the binder solution to obtain drug layered pellets;

(d) drying the pellets;

(e) optionally, subcoating the drug layered pellets;

(f) coating the pellets of (d) or (e) with a pH dependent polymer or a release controlled polymer; and

(g) filling the coated pellets that are obtained into capsules.

In embodiments, stabilized modified release formulations further comprise a dissolution enhancer for the drug.

In embodiments, the application relates to stabilized modified release formulations of dexlansoprazole, wherein concentrations of a dissolution enhancer are in the range of about 0.1% to 5% by weight of the total composition.

In embodiments, a dissolution enhancer for the drug is sodium chloride.

In embodiments, a dissolution enhancer also acts as a stabilizer for the drug.

In embodiments, the dissolution enhancer may be present in the subcoating or intermediate layer.

In embodiments, the application includes methods of stabilizing formulations of dexlansoprazole, comprising:

(a) layering a suspension, dispersion, or solution of dexlansoprazole, at least one stabilizer, and a dissolution enhancer, optionally together with one or more pharmaceutically acceptable excipients, onto a particulate pharmacologically inert substance, followed by drying;

(b) optionally, applying an intermediate coating and drying;

(c) applying a pH-dependently soluble release controlled coating layer surrounding the core of (a) or intermediate layer of (b);

(d) optionally, applying a release-controlled coating layer over (c), wherein the release-controlled coating layer comprises a diffusion-controlled polymer, gel-forming polymer, extended-release polymer, or any combination thereof; and

(e) combining the formed composition with at least one pharmaceutically acceptable excipient.

In embodiments, capsules comprise at least two fractions of coated pellets, wherein one fraction is coated with one or more release-controlled polymers and the other fraction is coated with one or more pH dependent polymers. In embodiments, capsules comprise at least two fractions of coated pellets, wherein both the fractions are coated with one or more release-controlled polymers, extended release polymers, or one or more pH dependent polymers.

The different physicochemical properties of the active ingredients, and as well as of the excipients, are to be considered, as these properties affect the process and formulation properties of the compositions. Various important physicochemical properties include, but are not limited to, particle sizes, density (bulk density and tapped density), compressibility index, Hausner's ratio, angle of repose, etc.

Particle sizes of an active pharmaceutical ingredient can affect the solid dosage form in numerous ways. For example, content uniformity (CU) of pharmaceutical dosage units can be affected by particle sizes and size distribution. This can be critical for low-dose drugs and satisfactory dosage units of low doses frequently cannot be manufactured from a drug that does not meet certain particle size and size distribution specifications. Also particle sizes play an important role in the dissolution of active ingredient from the final dosage form for certain drugs like dexlansoprazole, because of their poor solubility. Hence, these physicochemical properties not only affect the process of the preparing the pharmaceutical compositions but also affect the performance of the pharmaceutical product both in vitro and in vivo.

The selection of appropriate particles size distributions of dexlansoprazole, as well as of excipients, is within the scope of the disclosure. The D₁₀, D₅₀, and D₉₀ values are useful ways for indicating a particle size distribution. D₉₀ is the size value where at least 90 volume percent of the particles have sizes smaller than the given value. Likewise D₁₀ refers to 10 volume percent of the particles having sizes smaller than the given value. D₅₀ refers to at least 50 volume percent of the particles having sizes smaller than the given value, and D_{[4,3]} value refers to a mean particle size. Methods for determining D₁₀, D₅₀ D₉₀ and D_{[4,3]} include laser diffraction techniques, such as using equipment sold by Malvern Instruments Ltd., Malvern, Worcestershire, United Kingdom, or by Horiba.

In embodiments, compositions of the present disclosure comprise dexlansoprazole or a pharmaceutically acceptable form of dexlansoprazole having a particle size distribution such that D₉₀ is about 0.1 μm to about 1000 μm, or about 1 μm to about 500 μm, and D₅₀ is from about 0.01 μm to about 500 μm.

Flowability of materials is measured and represented using the Carr Index. The Carr Index is the percent ratio of the difference between tapped density and bulk density, to tapped density, mathematically described as:

Carr Index=[(Tapped density-Bulk density)÷Tapped density]×100.

The densities can be determined using the standard test method 616 (Bulk Density and Tapped Density) of United States Pharmacopeia 24, United States Pharmacopeial Convention, Inc., Rockville, Md., U.S.A., 1999.

Carr Index values below about 15% represent materials with very good flow properties and values above about 40% represent materials with very poor flow properties. Dexlansoprazole compositions in the form of multi-particulates such as granules or pellets of the present disclosure have Carr Index values substantially lower than 40%. Values for Carr Index for dexlansoprazole multi-particulate compositions of the disclosure are generally less than about 40%, or less than about 30%, or less than about 25%. This indicates superior handling capabilities during processing into pharmaceutical dosage forms.

In embodiments, the dexiansoprazole used to make the compositions, or contained in the compositions, is in amorphous or crystalline forms, or mixtures thereof. In embodiments, the dexlansoprazole used as the input active agent is in a substantially amorphous form, which form is substantially retained during the manufacturing of the composition and also during storage for commercially relevant times. In embodiments, the dexlansoprazole used as the input active agent is in a substantially crystalline form, which form is substantially retained during the manufacturing of the composition and also during storage for commercially relevant times. In embodiments, the dexlansoprazole used as the input active agent is in a substantially crystalline form, which form is converted into a substantially amorphous form during the manufacturing of the composition and which form remains substantially amorphous during storage of dosage forms for commercially relevant times.

An aspect of the present invention provides stabilized modified release pharmaceutical formulations of dexiansoprazole, wherein the polymorphic stability of dexiansoprazole is maintained during processing and storage.

In embodiments, the dexlansoprazole used as the input active agent is in a substantially amorphous form, wherein said form is substantially retained during the manufacturing of the formulation and also during storage of a dosage form for commercially relevant periods.

The compositions of the disclosure can be further processed into various pharmaceutical dosage forms as prepared, or can be combined with one or more pharmaceutically acceptable excipients. Different pharmaceutical dosage forms comprising the pharmaceutical compositions of the present disclosure include: solid oral dosage forms such as, but not limited to, powders, granules, pellets, tablets, and capsules. The modified release compositions may comprise hydrophilic, lipophilic, or hydrophobic release rate controlling substances, or their combinations to form matrix or reservoir, or combinations of matrix and reservoir systems. The compositions may be prepared using any techniques, such as one or more of direct blending, dry granulation, wet granulation, or extrusion and spheronization. Compositions may be presented as uncoated, film coated, sugar coated, powder coated, pH-dependently soluble release controlled coated, or modified release coated forms.

In embodiments, the disclosure includes oral pharmaceutical compositions in a solid dosage form, comprising: (a) a core containing dexlansoprazole, which is substantially free of basic substances; (b) a subcoating coated onto the core; and (c) a pH-dependently soluble release-controlled coating coated onto the subcoating. In certain embodiments, the subcoating is chemically inert.

In embodiments, the disclosure includes oral pharmaceutical compositions in a solid dosage form, comprising: (a) a core containing dexlansoprazole, which is substantially free of basic substances; (b) a subcoating coated onto the core; (c) a pH-dependently soluble release-controlled coating coated onto the subcoating; and (d) a release-controlled coating layer coated over pH-dependently soluble release-controlled coating (c). In certain embodiments, the subcoating is chemically inert.

In embodiments, the disclosure includes oral pharmaceutical compositions in a solid dosage form, comprising: (a) a core containing dexlansoprazole and a basic stabilizer; and (b) a pH-dependently soluble release-controlled coating layer. In embodiments, a pH-dependently soluble release-controlled coating layer is coated directly onto the core. In embodiments, the oral pharmaceutical compositions further include a subcoating coated onto the core, with the pH-dependently soluble release-controlled coating layer coated onto the subcoating layer.

Cores may include pharmaceutically acceptable excipients, such as surfactants, disintegrants, alkalizers, stabilizers, pH dependent or pH independent polymers, and/or binders. Cores of the present disclosure may be prepared by homogenously mixing dexlansoprazole and one or more pharmaceutically acceptable excipients, such as those mentioned hereinabove. In embodiments, cores of the present disclosure comprise inert materials such as diluent or sugar spheres, or cellulose spheres, onto which a solution or powder containing dexlansoprazole is sprayed or layered. The mixture is then formulated into small beads, pellets, granules, fine granules, or mini-tablets and filled into hard gelatin or soft gelatin capsules, such as by conventional procedures.

An inert subcoating can be used to separate a core from a pH-dependently soluble release controlled coating polymer that contains free carboxyl groups and may cause degradation and/or discoloration of an active agent. The inert subcoating may also serve as a pH-buffering zone, in which hydrogen ions diffusing from the outside toward the more alkaline core can react with hydroxyl ions diffusing from the alkaline core toward the surface of the coated articles. A subcoating may comprise one or more layers.

An inert subcoating can be applied to core pellets or mini-tablets using conventional coating procedures in a suitable coating pan or in fluidized bed apparatus, using water and/or organic solvents for the coating solutions or dispersions. Water soluble or insoluble polymers that can be used for an inert subcoating include, for example, sugars, zein, hydroxypropyl celluloses, hydroxypropyl methylcelluloses, hydroxyethyl celluloses, polyvinyl alcohols, providones, polyethylene glycols, poloxamers, ethyl celluloses, gelatin, polylysine, polyarginine, polyglycine, polyvinylpyrrolidine, vinyl acetate copolymers, and any mixtures thereof.

In embodiments, a drug stabilizer may be present in a subcoating.

In embodiments, a dissolution enhancer may be present in a subcoating.

In the case of mini-tablets, coatings may also be applied using a dry coating technique. An inert subcoating may also include pharmaceutically acceptable water-soluble or tablet excipients that rapidly disintegrate in water. Ordinary plasticizers, pigments, titanium dioxide, talc, and other additives may also be included in an inert subcoating. In the cases where gelatin capsules are used, the gelatin capsule itself can serve as a subcoating. The quantity of the inert subcoating of the present disclosure may vary from about 0.3% to 8%, or about 0.5 to 4% of the total weight of a core.

A pH-dependently soluble release-controlled coating is applied either directly onto cores, or onto subcoated cores, or over a drug layer, by conventional coating techniques such as, for instance, pan coating or fluidized bed coating using solutions of pH dependent polymers in water and/or suitable organic solvents, or by using suspensions of polymers, to provide a modified release of the active agent. pH-dependently soluble release-controlled coating polymers that can be used, for example, include cellulose acetate phthalates (CAP), hydroxypropyl methylcellulose phthalates (HPMCP), polyvinyl acetate phthalates (PVAP), hydroxypropyl methylcellulose acetate succinates (HPMCAS), cellulose acetate trimellitates, hydroxypropyl methylcellulose succinates, cellulose acetate succinates, cellulose acetate hexahydrophthalates, cellulose propionate phthalates, copolymers of methylmethacrylic acid and methyl methacrylate, copolymers of methyl acrylate, methylmethacrylate and methacrylic acid, copolymers of methylvinyl ether and maleic anhydride (e.g., Gantrez™ ES series), natural resins such as zein, shellac, and copal collophorium, carboxymethyl ethylcelluloses, co-polymerized methacrylic acid/methacrylic acid methyl esters such as, for instance, materials sold by Evonik Industries, Germany under the trade name EUDRAGIT® L12.5, L100, or EUDRAGIT® S12.5, S100, and several commercially available pH-dependently soluble release controlled dispersion systems (e.g., EUDRAGIT® L30D55, EUDRAGIT® FS30D, EUDRAGIT® L100-55, and EUDRAGIT® S100), KOLLICOAT® MAE30D and 30DP (from BASF), ESTACRYL® 30D (from Eastman Chemical), AQUATERIC® and AQUACOAT® CPD30 (from FMC), and mixtures thereof. The pH-dependently soluble release-controlled coating polymers may be used alone or as a mixture of at least two by mixing at in appropriate proportions to achieve dissolution in the desired pH ranges, for example pH ranges of 3-7, 4-8, 5-6, 5-7, 6-6.5, 6.5-8, 5-8, and 6-8.

In embodiments, the present disclosure relates to stabilized modified release pharmaceutical formulations, wherein a pH-dependently soluble release-controlled coating comprises hydroxypropyl methylcellulose phthalate.

The pH-dependently soluble release-controlled coating layer can optionally contain a pharmaceutically acceptable plasticizer such as, for instance, cetanol, triacetin, citric acid esters such as triethyl citrate, for instance, products sold under the trade name Citroflex® (from Vertellus Specialties, Inc., Indianapolis USA), phthalic acid esters, dibutyl succinate, and similar plasticizers. The amount of plasticizer is usually optimized for each pH-dependently soluble release controlled coating layer polymer and is usually in the range of about 1-40% of the pH-dependently soluble release controlled coating polymer. Dispersants such as talc, colorants and pigments may also be included into a pH-dependently soluble release controlled coating layer. The weight of pH-dependently soluble release controlled coating layer applied usually is about 1-20%, or about 2-10% of the weight of core material or the subcoated core material.

In embodiments, a coating is applied either directly onto cores or onto subcoated cores, by conventional coating techniques such as, for instance, pan coating or fluidized bed coating using pH dependent polymers dissolved or dispersed in water and/or suitable organic solvents, or by using suspensions of said polymers, to provide a modified release of the active agent.

In embodiments, the cores contain one or more release modifying polymers and/or lipophilic substances in admixture with dexlansoprazole to form a matrix. In certain embodiments, a modified release matrix is further coated with pH dependent polymers or release controlled polymers, or combinations thereof.

One or more polymers that can be used in a release-controlled coating layer of the present disclosure for modified release include pH-independent polymers such as ‘diffusion-controlled polymers’, or ‘gel-forming polymers’, or ‘eroding or disintegrating polymers’, or ‘matrix-forming polymer’, or ‘extended-release polymer’, or any combination thereof. Examples of polymers include, without limitation thereto, carbomers, polyamides, polycarbonates, polyalkylenes, polyalkylene glycols, polyalkylene oxides, polyalkylene terepthalates, polyvinyl ethers, polyvinyl esters, polyvinyl halides, polyvinyl acetates, polyglycolides, polysiloxanes, polyurethanes and copolymers thereof, alkyl celluloses, hydroxyalkyl celluloses, cellulose ethers, cellulose esters, nitrocelluloses, methyl celluloses, ethyl celluloses, hydroxypropyl celluloses, hydroxypropyl methylcelluloses, hydroxyethylcellulose, ethylcellulose, carboxymethylcellulose sodium, polyvinylpyrrolidones, including non-crosslinked polyvinylpyrrolidones, carboxymethylstarch, polyethylene glycols, polyoxyethylenes, poloxamers (polyoxyethylene-polyoxypropylene copolymers), polyvinylalcohols, natural polymers such as alginates and other polysaccharides that include, but are not limited to, arabinans, fructans, fucans, galactans, galacturonans, glucans, mannans, xylans (such as, for example, inulin), levan, fucoidan, carrageenan, galatocarolose, pectic acid, pectin, amylose, pullulan, glycogen, amylopectin, celluloses, dextran, pustulan, chitin, chitosan, agarose, keratan, chondroitan, dermatan, hyaluronic acid, alginic acid, xanthan gum, guar gum, starchres, and various other natural homopolymer or heteropolymers such as those containing one or more of aldoses, ketoses, acids or amines, erythrose, threose, ribose, arabinose, xylose, lyxose, allose, altrose, glucose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof, and including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, and other prolamines and hydrophobic proteins, synthetic polymers such as polymers of lactic acid and glycolic acid, polyanhydrides, poly (ortho)esters, polyurethanes, poly (butyric acid), poly (valeric acid), poly (caprolactone), poly (hydroxybutyrate), poly (lactide-co-glycolide), poly (lactide-co-caprolactone) copolymers, alpha-, beta- or gamma-cyclodextrins, dextrin derivatives (e.g. dextrin), ethyl acrylate-methyl methacrylate-trimethyl ammonium ethyl methacrylate chloride copolymers—Eudragit® RS (aminoalkylmethacrylate copolymer RS) or Eudragit® RL (aminoalkylmethacrylate copolymer RL), methyl methacrylate-ethyl acrylate copolymers (Eudragit® NE30D), cellulose acetates, cellulose butyrates, cellulose diacetates, cellulose triacetates, cellulose propionates, cellulose acetate butyrates, and other acetylated cellulose derivatives, waxes (e.g., carnauba wax, microcrystalline wax, beeswax, and polyethoxylated beeswax), natural fats (coconut, soya, cocoa) including modified forms such as totally or partially hydrogenated, hydrogenated castor oil, hydrogenated vegetable oil, and fatty acid derivatives such as mono-, bi- and tri-substituted glycerides, phospholipids, glycerophospholipids, glyceryl palmitostearate, glyceryl behenate, glyceryl monostearate, diethyleneglycol palmitostearate, polyethyleneglycol stearate, polyethyleneglycol palmitostearate, polyoxyethylene-glycol palmitostearate, glyceryl monopalmitostearate, cetyl palmitate, fatty alcohols associated with polyethoxylate fatty alcohols, cetyl alcohol, stearic acid, saturated or unsaturated fatty acids and their hydrogenated derivatives, lecithin, cephalins, chitosan and derivatives thereof, sphingolipids, sterols such as cholesterol and its substituted derivatives, polyethylene oxide (PEO), for example, Polyox® WSR 303, molecular weight: 7000000, Polyox® WSR Coagulant, molecular weight: 5000000, Polyox® WSR 301, molecular weight: 4000000, Polyox® WSR N-60K, molecular weight: 2000000, and Polyox® WSR 205, molecular weight: 600000; manufactured by Dow Chemical Co., Ltd., hydroxypropyl methylcelluloses (HPMC), such as Metlose® 90SH10000, Metlose® 90SH50000, and Metlose® 90SH30000, manufactured by Shin-Etsu Chemical Co., Ltd., carboxymethylcelluloses (CMC-Na), such as Sanlose F-1000MC, hydroxypropyl celluloses (HPC), for example, HPC-H, manufactured by Nippon Soda Co., Ltd., hydroxyethyl cellulose (HEC), such as carboxyvinyl polymer HIVISWAKO 103, 104 and 105 manufactured by Wako Pure Chemical Industries Ltd.; CARBOPOL 943 manufactured by Goodrich Co., Ltd., and the like, including mixtures thereof. These may be used alone or as a mixture of at least 2 by mixing at in appropriate proportions. Further, these materials may be mixed in an appropriate ratio, and can be used by mixing with hydrophilic pore forming substances such as hydroxypropyl methylcelluloses (HPMC), hydroxypropylcelluloses (HPC), carboxyvinyl polymers, polyethylene glycols (e.g., PEG 6000), lactose, mannitol, and organic acids. Optionally, the release controlled layer may further comprise one or more pH-dependently soluble release controlled polymers.

In an embodiment, the present disclosure relates to stabilized modified release pharmaceutical formulations, wherein a release-controlled coating layer comprises one or more pH-dependently soluble release-controlled coating polymers.

In an embodiment, the present disclosure relates to stabilized modified release pharmaceutical formulations, wherein a release-controlled coating layer comprises poly(ethyl acrylate, methyl methacrylate, trimethylammonioethyl methacrylate chloride) and poly(methacrylic acid-ethyl acrylate).

In embodiments, multi-particulates are coated with a gel forming polymer release-controlled coating layer, wherein the gel forming polymer release-controlled coating layer comprises a gel forming polymer such as polyethylene oxide (PEO), for example, Polyox® WSR 303, molecular weight: 7000000, Polyox® WSR Coagulant, molecular weight: 5000000, Polyox® WSR 301, molecular weight: 4000000, Polyox®WSR N-60K, molecular weight: 2000000, and Polyox® WSR 205, molecular weight: 600000; manufactured by Dow Chemical Co., Ltd., hydroxypropyl methylcelluloses (HPMC), such as Metlose® 90SH10000, Metlose® 90SH50000, and Metlose® 90SH30000, manufactured by Shin-Etsu Chemical Co., Ltd., carboxymethylcelluloses (CMC-Na), such as Sanlose F-1000MC, hydroxypropyl celluloses (HPC), for example, HPC-H, manufactured by Nippon Soda Co., Ltd., hydroxyethyl cellulose (HEC), such as carboxyvinyl polymer HIVISWAKO 103, 104 and 105 manufactured by Wako Pure Chemical Industries Ltd.; CARBOPOL 943 manufactured by Goodrich Co., Ltd., chitosan, sodium alginate, pectin and the like. These may be used alone or as a mixture of at least 2 by mixing in appropriate proportions.

In embodiments, multiparticulates are coated with a diffusion-controlled release coating layer, wherein the diffusion-controlled release coating layer comprises a diffusion controlling polymer such as ethyl acrylate-methyl methacrylate-trimethylammoniumethyl methacrylate chloride copolymer (Eudragit® RS (aminoalkylmethacrylate copolymer RS) or Eudragit® RL (aminoalkylmethacrylate copolymer RL), manufactured by Evonik Industries, methyl methacrylate-ethyl acrylate copolymer (Eudragit® NE30D), ethyl cellulose, and the like. Further, mixtures of these may be used in appropriate ratios, and they can be used by mixing with hydrophilic pore forming substances such as hydroxypropyl methylcellulose (HPMC), hydroxypropylcellulose (HPC), carboxyvinyl polymers, polyethylene glycols such as PEG 6000, lactose, mannitol, and organic acids.

In embodiments, the present disclosure relates to stabilized modified release pharmaceutical formulations, wherein a stabilizer in a drug layer is a water-soluble excipient.

Various useful stabilizers according to the present disclosure include, water soluble polymers such as, but not limited to, polyvinylpyrrolidones or povidones (such as grades PVP-K25, PVP-K29, PVP-K30, and PVP-K90), hydroxypropyl celluloses, hydroxyethyl celluloses, hydroxypropyl methylcelluloses, polyvinyl alcohols, carboxymethylcellulose sodium, and any mixtures thereof. Further water soluble excipients according to the present application include sugars and sugar alcohols, preferably having low hygroscopicity, and include, for example, mannitol, lactose, fructose, sorbitol, xylitol, maltodextrin, dextrates, dextrins, lactitol, and mixtures of any two or more thereof.

In embodiments, the application relates to stabilized modified release formulations of dexlansoprazole, wherein concentrations of stabilizer are in the range of about 0.001% to 10% by weight of the total composition.

In embodiments, the present application relates to stabilized modified release formulations of dexlansoprazole, wherein a stabilizer is a polyvinylpyrrolidone.

In embodiments, stabilized modified release pharmaceutical formulations of the present disclosure further comprise a drug dissolution enhancer.

Dissolution enhancers increase the rate of dissolution of the drug from the carrier. In general, dissolution enhancers are amphiphilic compounds and are generally more hydrophilic than the carrier. Exemplary dissolution enhancers include: salts such as sodium chloride, potassium chloride, lithium chloride, calcium chloride, magnesium chloride, sodium sulfate, sodium acetate, potassium sulfate, sodium carbonate, magnesium sulfate, and potassium phosphate; alcohols such as stearyl alcohol, cetyl alcohol, and polyethylene glycol; surfactants, such as poloxamers (such as poloxamer 188, poloxamer 237, poloxamer 338, and poloxamer 407), docusate salts, polyoxyethylene alkyl ethers, polyoxyethylene castor oil derivatives, polysorbates, polyoxyethylene alkyl esters, sodium lauryl sulfate, and sorbitan monoesters; sugars and sugar alcohols such as mannitol, glucose, sucrose, xylitol, sorbitol, and maltitol; amino acids such as alanine and glycine; and any mixtures thereof.

In embodiments, the application relates to stabilized modified release formulations of dexlansoprazole, wherein concentrations of a dissolution enhancer are in the range of about 0.1% to 5% by weight of the total composition.

In embodiments, a stabilizer and/or dissolution enhancer may be present in the subcoating.

In embodiments, dexlansoprazole is used for preparing inclusion complexes of drug with a cyclodextrin. In embodiments, an amorphous form of dexlansoprazole is used for preparing inclusion complexes with cyclodextrins.

As used herein, “cyclodextrin” refers to any of the natural cyclodextrins, α-cyclodextrin, β-cyclodextrin, and y-cyclodextrin, and their respective derivatives or analogs. Cyclodextrins (sometimes called cycloamyloses) make up a family of cyclic oligosaccharides, composed of 5 or more α-D-glucopyranoside units linked 1-4, as in amylose (a fragment of starch). The formation of the inclusion complexes greatly modifies the physical and chemical properties of the guest molecules (such as dexlansoprazole in the present disclosure), mostly in terms of aqueous solubility. An inclusion complex of dexlansoprazole with cyclodextrins also aids in penetration of the drug into body tissues.

Any cyclodextrin, which enhances the aqueous solubility and/or provides for effective delivery of dexlansoprazole, may be used in the present disclosure. The cyclodextrins of the present disclosure can include the natural occurring cyclodextrins and their derivatives. The natural cyclodextrins include α-cyclodextrin, β-cyclodextrin, and y-cyclodextrin. Derivatives are typically prepared by modifying the hydroxyl groups located on the exterior or hydrophilic side of the cyclodextrin molecule. The modifications can be made to increase the aqueous solubility and the stability of the complexes and can modify the physical characteristics of complexes, including the formation and dissociation of the complexes. The types and degrees of modification, as well as their preparation, are well-known in the art.

Any of the natural cyclodextrins can be derivatized, such as derivatives of β-cyclodextrin. Cyclodextrin derivatives include alkylated cyclodextrins, comprising methyl-, dimethyl-, trimethyl-, and ethyl-3-cyclodextrins; hydroxyalkylated cyclodextrins, including hydroxyethyl-, hydroxypropyl-, and dihydroxypropyl-β-cyclodextrin, ethylcarboxymethyl cyclodextrins, sulfate, sulfonate and sulfoalkyl cyclodextrins, such as β-cyclodextrin sulfate, β-cyclodextrin sulfonate, and β-cyclodextrin sulfobutyl ether, as well as polymeric cyclodextrins. Other cyclodextrin derivatives can be made by substitution of the hydroxy groups with saccharides, such as glucosyl- and maltosyl-β-cyclodextrin.

Other cyclodextrins include the naturally occurring cyclodextrins, methyl-β-cyclodextrin, dimethyl-β-cyclodextrin, trimethyl-β-cyclodextrin, 2-hydroxymethyl-β-cyclodextrin, hydroxyethyl-β-cyclodextrin, 2-hydroxypropyl-β-cyclodextrin, β-hydroxypropyl-β-cyclodextrin, β-cyclodextrin sulfate, β-cyclodextrin sulfonate, and β-cyclodextrin sulfobutyl ether. Any of the above cyclodextrins or their derivatives or polymers prepared from them can be used for preparation of the compositions of the disclosure, either alone or in the form of mixtures of one or more cyclodextrins.

In embodiments, pharmaceutical compositions of the present disclosure comprise dexlansoprazole, adsorbed onto at least one pharmaceutically acceptable carrier. Useful carriers according to the present disclosure include, but are not limited to, polyvinylpyrrolidones, hydroxypropyl methylcelluloses, sugar alcohols such as mannitol, sorbitol, etc., and the like.

In embodiments, the disclosure includes modified release pharmaceutical compositions comprising dexlansoprazole, optionally together with one or more pharmaceutically acceptable excipients, wherein said compositions are in multi-particulate form.

In embodiments, the disclosure includes modified release pharmaceutical compositions comprising cores comprising dexlansoprazole, optionally together with one or more pharmaceutically acceptable excipients, and a coating comprising one or more polymers, wherein the compositions are in multi-particulate form.

In embodiments, modified release dexlansoprazole-containing multi-particulates comprise non-parielo cores such as inert sugar or similar substances, upon which dexlansoprazole is loaded, optionally together with one or more pharmaceutically acceptable excipients, using any techniques such as powder layering, solution spraying, suspension spraying, or any other technique known to the art.

In embodiments, modified release compositions of the disclosure comprise dexlansoprazole loaded non-pariel cores having a coating comprising one or more release controlled polymer, pH dependent polymer, or combinations thereof.

In embodiments, the disclosure includes pharmaceutical compositions comprising modified release multi-particulates comprising dexlansoprazole, comprising dexlansoprazole-containing cores and a coating comprising one or more polymers, and optionally having one or more further coatings.

In embodiments, multi-particulates comprising dexlansoprazole further contain one or more non-functional coating or functional coating, to provide modified release of the active agent.

Multi-particulate formulations of the disclosure can be prepared using the techniques described herein, as well as other methods known to those having skill in the art.

In embodiments, multi-particulates comprising dexlansoprazole are coated with varying amounts of polymers, giving fractions having different release profiles, and these can be combined to form a pharmaceutical composition or dosage form to achieve desired modified release profiles.

In embodiments, multi-particulates comprising dexlansoprazole are coated with different types of polymers, for example pH-dependently soluble release controlled coating polymers (pH dependent polymers) and modified release polymers (release controlled polymers), giving different release profiles, and these can be combined to form a pharmaceutical composition or dosage form to achieve desired modified release profiles.

In embodiments, multi-particulates comprising dexlansoprazole can be combined with pharmaceutically acceptable excipients, and compounded to form a pharmaceutical composition, which can be compressed into tablets or placed into suitable capsule shells, using techniques known to those having skill in the art. In embodiments, compositions of the present disclosure are filled into hard gelatin capsules, wherein empty hard gelatin capsule shells comprise one or more of hydroxymethyl cellulose, carrangeenan, potassium chloride, polyvinyl polymers such as polyvinyl acetate and polyvinyl alcohol, and the like.

Pharmaceutically acceptable excipients according to the present disclosure include, for example, any one or more of diluents, binders, alkalizers, lubricants, glidants, disintegrating agents, surfactants, and other additives that are commonly used in solid pharmaceutical dosage form preparations.

Various useful fillers or diluents according to the present disclosure include, but are not limited to, starches, lactose, mannitol (e.g., Pearlitol™ SD200), cellulose derivatives, confectioner's sugar, and the like. Different grades of lactose include, but are not limited to, lactose monohydrate, lactose DT (direct tableting), lactose anhydrous, Flowlac™ (available from Meggle Products), Pharmatose™ (available from DMV), and others. Different starches include, but are not limited to, maize starch, potato starch, rice starch, wheat starch, pregelatinized starch (commercially available as PCS PC10 from Signet Chemical Corporation), starch 1500, starch 1500 LM grade (low moisture content grade) from Colorcon, fully pregelatinized starch (commercially available as National 78-1551 from Essex Grain Products), and others. Different cellulose compounds that can be used include crystalline cellulose and powdered cellulose. Examples of crystalline cellulose products include, but are not limited to, CEOLUS™ KG801, Avicel™ PH101, PH102, PH301, PH302 and PH-F20, PH-112, microcrystalline cellulose 114, and microcrystalline cellulose 112. Other useful diluents include, but are not limited to, carmellose, sugar alcohols such as mannitol, sorbitol and xylitol, calcium carbonate, magnesium carbonate, dibasic calcium phosphate, and tribasic calcium phosphate.

Various useful binders according to the present disclosure include, but are not limited to, hydroxypropylcelluloses, also called HPC (e.g., Klucel™ LF, Klucel EXF) and useful in various grades, hydroxypropyl methylcelluloses, also called hypromellose or HPMC (e.g., Methocel™ products) and useful in various grades, polyvinylpyrrolidones or povidone (such as grades PVP-K25, PVP-K29, PVP-K30, and PVP-K90), copovidone (e.g., Plasdone™ S 630), powdered acacia, gelatin, guar gum, carbomers (e.g., a Carbopol™ product), methylcelluloses, polymethacrylates, and starches.

Various useful disintegrants include, but are not limited to, carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™ from FMC-Asahi Chemical Industry Co., Ltd.), crospovidones, examples of commercially available crospovidone products including but not limited to crosslinked povidone, Kollidon™ CL from BASF (Germany), Polyplasdone™ XL, XI-10, and INF-10 from ISP Inc. (USA), and low-substituted hydroxypropylcelluloses. Examples of low-substituted hydroxypropylcelluloses include, but are not limited to, low-substituted hydroxypropylcellulose LH11, LH21, LH31, LH22, LH32, LH20, LH30, LH32 and LH33 (all from Shin-Etsu Chemical Co., Ltd.). Other useful disintegrants include sodium starch glycolate, colloidal silicon dioxide, and starches.

Alkalizers that are useful in present disclosure include, but are not limited to, basic inorganic salts and organic compounds. Various useful basic inorganic salts include, but are not limited to, basic inorganic salts of sodium, potassium, magnesium, and calcium. Examples of the basic inorganic salts of sodium are sodium carbonate, sodium hydrogen carbonate, sodium hydroxide, and the like. Examples of the basic inorganic salts of potassium are potassium carbonate, potassium hydrogen carbonate, potassium hydroxide, and the like. Examples of basic inorganic salts of magnesium are heavy magnesium carbonate, magnesium carbonate, magnesium oxide, magnesium hydroxide, magnesium metasilicate aluminate, magnesium silicate, magnesium aluminate, synthetic hydrotalcite [Mg₆Al₂(OH)₁₆.CO₃.4H₂O], aluminum hydroxide-magnesium [2.5MgO.Al₂O₃.xH₂O], and the like. Examples of basic inorganic salts of calcium include precipitated calcium carbonate, calcium hydroxide, and the like.

Organic bases that may be used in the present disclosure include pharmaceutically acceptable organic bases, such as, but not limited to, meglumine, lysine, N,N′-dibenzylethylenediamine, chloroprocain, choline, diethanolamine, ethylenediamine, procaine, and mixtures of any two or more thereof.

In embodiments, the disclosure relates to stabilized modified release formulations of dexlansoprazole, wherein concentrations of alkalizer are in the range of about 0.1% to about 10%, by weight of the total composition.

In embodiments, the application relates to stabilized modified release formulations of dexlansoprazole, wherein weight ratios of dexlansoprazole to alkaline compound are in the range of about 1:0.01 to 1:5.

Useful surface-active agents according to the present disclosure include non-ionic, cationic, anionic, and zwitterionic surface-active agents. Useful non-ionic surface-active agents include ethylene glycol stearates, propylene glycol stearates, diethylene glycol stearates, glycerol stearates, sorbitan esters (e.g., SPAN™ products) and polyhydroxyethylenically treated sorbitan esters (e.g., TWEEN™ products), aliphatic alcohols, phenol, and PEG ethers. Useful cationic surface-active agents include quaternary ammonium salts (e.g. cetyltrimethylammonium bromide) and amine salts (e.g. octadecylamine hydrochloride). Useful anionic surface-active agents include sodium stearate, potassium stearate, ammonium stearate, and calcium stearate, triethenolamine stearate, sodium lauryl sulphate, sodium dioctylsulphosuccinate, and sodium dodecylbenzenesulphonate. Natural surface-active agents may also be used, such as for example phospholipids, e.g. diacylphosphatidyl glycerols, diaceylphosphatidyl cholines, and diaceylphosphatidic acids, the precursors and derivatives thereof, such as for example soybean lecithin and egg yolk.

Useful lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and combinations thereof.

One or more glidant materials, which improve the flow of powder blends, pellets, or mini-tablets and minimize dosage form weight variations can be used. Useful glidants include but are not limited to silicone dioxide, talc, and combinations thereof.

Colouring agents can be used to colour code the compositions, for example, to indicate the type and dosage of the therapeutic agent therein. Colouring agents can also be used to differentiate the varied fractions of multi-particulates comprised in a unit dosage form such as a capsule. Suitable colouring agents include, without limitation, natural and/or artificial compounds such as FD&C colouring agents, natural juice concentrates, pigments such as titanium oxide, silicon dioxide, iron oxides, zinc oxide, combinations thereof, and the like.

Various solvents can be used in the processes of preparation of pharmaceutical compositions of the present disclosure, including but not limited to water, methanol, ethanol, dichloromethane, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran and mixtures thereof.

In embodiments, compositions of the present disclosure contain at least one antioxidant. The antioxidant may be present either as a part of the composition or as a packaging component. Thus, in a particular embodiment of processes according to the present disclosure, an antioxidant is introduced into the formulation during the drug loading stage over inert cores. The antioxidants are present in an amount effective to retard decomposition of dexlansoprazole, as it is susceptible to oxidation.

In embodiments, the content of antioxidant in the formulation ranges from about 0.001 to 10 weight percent, with respect to the active agent content.

Among the antioxidants, non-limiting examples include ascorbic acid and its salts, tocopherols, sulfite salts, such as sodium metabisulfite or sodium sulfite, sodium sulfide, dl-alpha-tocopherol, butylated hydroxyanisole, butylated hydroxytoluene, ascorbyl palmitate, and propyl gallate. Other suitable antioxidants will be readily recognized by those skilled in the art.

Useful additives for coatings include, but are not limited to, any one or more of plasticizers, antiadherents, opacifiers, solvents, colorants, lubricants, pigments, antifoam agents, and polishing agents.

Various useful plasticizers include, but are not limited to, substances such as castor oil, diacetylated monoglycerides, dibutyl sebacate, diethyl phthalate, glycerin, polyethylene glycol, propylene glycol, triacetin, and triethyl citrate. Also, mixtures of plasticizers may be utilized. The type of plasticizer depends upon the type of coating agent. An opacifier like titanium dioxide may also be present in an amount ranging from about 10% to about 20% based on the total weight of the coating.

Antiadherents are frequently used in film coating processes to avoid sticking effects during film formation and drying. An example of a useful substance for this purpose is talc. The antiadherent is frequently present in the film coating in an amount of about 5% (w/w) to 15% (w/w), based upon the total weight of the coating.

The foregoing descriptions of excipients are not intended to be exhaustive. Those skilled in the art will be aware of many other substances that are useful in the practice of the disclosure, and the use of such substances is specifically included in this disclosure.

In an aspect, the disclosure includes methods of preparing the pharmaceutical compositions of the present disclosure.

Equipment suitable for processing the pharmaceutical compositions of the present disclosure include any one or more of rapid mixer granulators, planetary mixers, mass mixers, ribbon mixers, fluid bed processors, mechanical sifters, blenders, roller compacters, extrusion-spheronizers, compression machines, capsule filling machines, rotating bowls or coating pans, tray dryers, fluid bed dryers, rotary cone vacuum dryers, and the like, multimills, fluid energy mills, ball mills, colloid mills, roller mills, hammer mills, and the like, equipped with a suitable screen.

In embodiments, the disclosure includes packaging for the dexlansoprazole compositions which maintain stability during storage, transportation, and use. The stabilization of the dexlansoprazole composition of the present disclosure can be improved by using packaging inhibiting the permeation of oxygen and moisture, packaging having inert gases (namely, packages with air replaced with gases other than oxygen), vacuum packaging and packages containing a deoxidizer. Stabilization is improved by reducing oxygen with which the solid preparation is directly brought in contact, using these package forms. When a deoxidizer is enclosed, the pharmaceutical solid preparation can be packaged with an oxygen impermeable material, and then this is enclosed within other packaging.

In embodiments, stable compositions of the present disclosure include a desiccant and/or an oxygen absorbent as a component of packaging. A desiccant is a hygroscopic substance that induces or sustains a state of dryness (desiccation) in its local vicinity in a moderately well-sealed container. Commonly encountered pre-packaged desiccants are solids, and work through absorption or adsorption of water, or a combination of the two. Desiccants for specialized purposes may be in forms other than solid, and may work through other principles, such as chemical bonding of water molecules. Pre-packaged desiccants (such as pouches) are most commonly used to remove excessive humidity that would normally degrade or even destroy products sensitive to moisture. Non-limiting examples of various desiccants are anhydrous calcium sulfate (e.g., Drierite® products), silica gel, calcium chloride, montmorillonite clay, and molecular sieves. Commercially available oxygen absorbent products such as StabilOx® are useful in minimizing the degradation of active agent due to oxidation.

In embodiments, the disclosure includes the use of packaging materials such as containers and closures of high-density polyethylene (HDPE), low-density polyethylene (LDPE) and/or polypropylene and/or glass, and blisters or strips composed of moisture resistant aluminum, high-density polypropylene, polyvinyl chloride and/or polyvinylidene dichloride.

In embodiments, the disclosure includes forms of packaging for formulations of dexlansoprazole, such that retardation of drug release from the formulations is prevented. In embodiments, the disclosure provides a package suitable for commercial sale, which provides stability during storage, transportation, and use.

In embodiments, the pharmaceutical dosage forms of the present disclosure are intended for oral administration to a patient in need thereof.

In an aspect, this disclosure provides methods of treating gastrointestinal inflammatory diseases and gastric acid-related diseases in mammals and man including reflux esophagitis, gastritis, duodenitis, gastric ulcer, and duodenal ulcer, using the formulations and pharmaceutical compositions of the present disclosure. The compounds and compositions of this disclosure may be administered to a subject in a therapeutically effective amount.

Pharmaceutical dosage forms can be subjected to in vitro dissolution testing, such as according to Test 711 “Dissolution” in United States Pharmacopeia 29, United States Pharmacopeial Convention, Inc., Rockville, Md., 2005 (USP), to determine the rate at which the active substance is released from the dosage forms, and content of active substance can be determined in dissolution media using techniques such as high performance liquid chromatography (HPLC).

An example of a useful HPLC analytical method for determining dexlansoprazole concentrations and impurity contents is one using a Xterra RP18 column, 250×4.6 mm, 3.5 μm, and the following parameters:

Flow: 0.8 mL/minute.

Detector wavelength: 285 nm.

Column temperature: 25° C.

Injection volume: 10 μL.

Sample cooler temperature: 5° C.

Run time: 100 minutes.

Buffer: Dissolve about 6.8 g of potassium dihydrogen phosphate in HPLC grade water, add 10 mL of triethylamine, mix well, and dilute to 1000 mL. Adjust pH of the solution to 7.0 with orthophosphoric acid solution. Filter through a 0.45 μm nylon 66 membrane filter. Degas in a sonicator for 10 minutes.

Mobile phase A: Mix buffer and acetonitrile in the volume ratio 90:10 respectively. Degas in a sonicator for 10 minutes.

Mobile phase B: Mix acetonitrile and buffer in the volume ratio 70:30 respectively. Degas in a sonicator for 10 minutes.

Gradient elution program: as in Table 1.

TABLE 1

| Minutes | Mobile phase A (%) | Mobile phase B (%) |
|---|---|---|
| 0 | 90 | 10 |
| 50 | 40 | 60 |
| 67 | 20 | 80 |
| 85 | 20 | 80 |
| 90 | 90 | 10 |
| 100 | 90 | 10 |

The test solution is prepared in diluent (prepared by mixing 0.1 N sodium hydroxide and ethanol in a volume ratio of 3:5) and a 10 μL aliquot is injected into the column of a gradient high-performance liquid chromatography unit. Absorbance is monitored at 285 nm.

For the analysis, a blank is injected, followed by two injections of diluted standard, then a sample. Among the observed peaks are those having the relative retention times and relative response factors (dexlansoprazole=1) as shown in Table 2.

TABLE 2

| Impurity | RRT | RRF |
|---|---|---|
| A | 0.14 | 1.04 |
| B | 0.25 | 0.92 |
| C | 0.36 | 2.66 |
| D | 1.06 | 0.92 |
| E | 1.43 | 1.11 |
| F | 1.85 | 1.96 |
| G | 1.41 | 1.11 |
| H | 1.68 | 0.69 |

Certain specific aspects and embodiments of the disclosure will be explained in greater detail with reference to the following examples, being provided only for purposes of illustration, and it is to be understood that the present disclosure is not to be limited thereto.

### Examples 1-2

### Formulations for Dexlansoprazole 60 mg Capsules

| | mg/Capsule | | | |
|---|---|---|---|---|
| | Pellet | | Pellet | |
| | Fraction 1 | | Fraction 2 | |
| | Exam- | Exam- | Exam- | Exam- |
| Ingredient | ple 1 | ple 2 | ple 1 | ple 2 |

A. Seal Coating Layer

| | | | | |
|---|---|---|---|---|
| Sugar spheres (25-30 mesh) | 38 | 38 | 38 | 38 |
| Hydroxypropyl methylcellulose, | 2 | 2 | 2 | 2 |
| 5 cps | | | | |
| Methylene dichloride* | q.s. | q.s. | q.s. | q.s. |
| Methanol* | q.s. | q.s. | q.s. | q.s. |
| Pellet wt. | 40 | 40 | 40 | 40 |

B. Drug Layer

| | | | | |
|---|---|---|---|---|
| Dexlansoprazole (amorphous) | 15 | 15 | 45 | 45 |
| Magnesium oxide (light) | 10 | 10 | 30 | 30 |
| Polyvinylpyrrollidone K30 | 10 | 10 | 30 | 30 |
| Methylene chloride* | q.s | q.s | q.s. | q.s. |
| Methanol* | q.s. | q.s. | q.s. | q.s. |

C. Subcoating Layer

| | | | | |
|---|---|---|---|---|
| Hydroxypropyl methylcellulose, | 7.5 | 7.5 | 22.5 | 22.5 |
| 5 cps | | | | |
| Talc | 3 | 3 | 9 | 9 |
| Titanium dioxide | 4.5 | 4.5 | 13.5 | 13.5 |
| Isopropyl alcohol* | q.s. | q.s. | q.s. | q.s. |
| Methylene chloride* | q.s. | q.s. | q.s. | q.s. |

D. pH-Dependently Soluble Release-Controlled Coating Layer

| | | | | |
|---|---|---|---|---|
| Eudragit ® L100-55 | 11.25 | 11.25 | 67.50 | 67.50 |
| Triethyl citrate | 1.13 | 1.13 | 6.75 | 6.75 |
| Talc | 5.63 | 5.63 | 33.75 | 33.75 |
| Methanol* | q.s. | q.s. | q.s. | q.s. |

E. Release-Controlled Coating Layer

| | | | | |
|---|---|---|---|---|
| Eudragit ® RLPO | — | — | 36.6 | — |
| Eudragit ® L100-55 | — | — | 9.1 | — |
| High viscosity hydroxypropyl | — | — | — | 45.7 |
| methylcellulose | | | | |
| Triethyl citrate | — | — | 6.9 | 6.9 |
| Talc | — | — | 23.0 | 23.0 |
| Methanol* | — | — | q.s. | q.s. |

*Evaporates during processing.

Manufacturing Procedure:

A. Seal Coating of Sugar Spheres

1. Hydroxypropyl methylcellulose (5 cps) was dissolved in a mixture of methanol and methylene chloride.

2. The solution was sprayed onto sugar spheres using a fluid bed processor (FBP), to achieve a weight gain after drying of 5%.

B. Drug Loading onto Sugar Spheres

1. Polyvinylpyrrollidone K30 was dissolved in a mixture of methanol and methylene chloride.

2. Magnesium oxide (light) was added to the solution and stirred to form dispersion.

3. Dexlansoprazole was dissolved in the dispersion, and the temperature of this solution was maintained at 2-15° C. throughout the process.

4. The drug solution was sprayed onto seal coated sugar spheres from step A2.

5. Coated particles were dried at 40° C. until loss on drying (LOD) was less than 2% w/w, measured at 105° C.

C. Subcoating layer

1. Hydroxypropyl methylcellulose (5 cps) was dissolved in a mixture of isopropyl alcohol and methylene chloride.

2. Talc and titanium dioxide were sifted through a 60 mesh sieve.

3. Talc and titanium dioxide were combined with a mixture of isopropyl alcohol and methylene chloride, and colloid milled.

4. Dispersion from step 3 was added to polymer solution of step 1 and stirred.

5. The dispersion of step 4 was applied to drug coated sugar spheres from step B5, using a fluid bed processor.

D. pH-Dependently Soluble Release-Controlled Coating Layer (Fraction 1)

1. Dissolved Eudragit® L100-55 in methanol with stirring.

2. Triethyl citrate was added to the solution with stirring.

3. Talc was added to the solution with stirring.

4. The dispersion of step 3 was sprayed onto 25% of the subcoated pellets from step C5, to achieve a weight gain after drying of 20%±3% w/w, using a FBP with bottom spray.

5. The dispersion of step 3 was sprayed onto 75% of the subcoated pellets from step C5, to achieve a weight gain after drying of 40%±3% w/w, using a FBP with bottom spray.

6. Coated pellets were dried in the FBP until LOD was between 1-3% w/w at 105° C., measured using a halogen moisture balance.

7. The pellets were cured in the FBP for 2 hours at 40° C.

E. Release-Controlled Coating Layer (Fraction 2)

1. Eudragit® RLPO and Eudragit® L100-55 were dissolved in methanol for Example 1 and High viscosity Hydroxypropyl methylcellulose was dissolved in methanol for Example 2.

2. Triethyl citrate was dissolved in the above solution of step 1.

3. Talc was added to solution of step 2, with stirring, and stirring was continued during the subsequent coating operation.

4. The dispersion from step 3 was sprayed onto 75% of the pH-dependently soluble release-controlled coated pellets from step D5, to achieve a weight gain after drying of 20%±5% w/w, using a FBP with bottom spray.

5. Coated pellets were dried in the FBP until LOD of the tablets were 1-3% w/w, measured at 105° C. using a halogen moisture balance.

6. The pellets were cured in the FBP for 2 hours at 40° C.

F. Encapsulation

1. Fraction 1 and 2 pellets were filled into capsules.

### Examples 3-5

### Formulations for Dexlansoprazole 60 mg Capsules

| | mg/Capsule | | | | | |
|---|---|---|---|---|---|---|
| | Pellet Fraction 1 | | | Pellet Fraction 2 | | |
| Ingredient | Example 3 | Example 4 | Example 5 | Example 3 | Example 4 | Example 5 |

A. Seal Coating Layer

| | | | | | | |
|---|---|---|---|---|---|---|
| Sugar spheres (25-30 | 38 | 38 | 38 | 38 | 38 | 38 |
| mesh) | | | | | | |
| Hydroxypropyl | 2 | 2 | 2 | 2 | 2 | 2 |
| methylcellulose, 5 cps | | | | | | |
| Methylene dichloride* | q.s. | q.s. | q.s. | q.s. | q.s. | q.s. |
| Methanol* | q.s. | q.s. | q.s. | q.s. | q.s. | q.s. |

B. Drug Layer

| | | | | | | |
|---|---|---|---|---|---|---|
| Dexlansoprazole | 15 | 15 | 15 | 45 | 45 | 45 |
| (amorphous) | | | | | | |
| Magnesium oxide (light) | 10 | 10 | 10 | 30 | 30 | 30 |
| Polyvinylpyrrollidone | 10 | 10 | 10 | 30 | 30 | 30 |
| K30 | | | | | | |
| Methylene chloride* | q.s | q.s | q.s | q.s. | q.s. | q.s. |
| Methanol* | q.s. | q.s. | q.s. | q.s. | q.s. | q.s. |

C. Sub-coating Layer

| | | | | | | |
|---|---|---|---|---|---|---|
| Hydroxypropyl | 7.5 | 7.5 | 7.5 | 7.5 | 22.5 | 22.5 |
| methylcellulose, 5 cps | | | | | | |
| Ethyl cellulose | — | — | — | 15 | — | — |
| Talc | 3 | 3 | 3 | 9 | 9 | 9 |
| Titanium dioxide | 4.5 | 4.5 | 4.5 | 13.5 | 13.5 | 13.5 |
| Isopropyl alcohol* | q.s. | q.s. | q.s. | q.s. | q.s. | q.s. |
| Methylene chloride* | q.s. | q.s. | q.s. | q.s. | q.s. | q.s. |

D. Release-Controlled Coating Layer

| | | | | | | |
|---|---|---|---|---|---|---|
| Eudragit ® RSPO | — | — | — | — | 36.6 | — |
| Eudragit ® L100-55 | — | — | — | — | 9.1 | — |
| High-viscosity | — | — | — | — | — | 45.7 |
| hydroxypropyl | | | | | | |
| methylcellulose | | | | | | |
| Triethyl citrate | — | — | — | — | 6.9 | 6.9 |
| Talc | — | — | — | — | 23 | 23 |
| Methanol* | — | — | — | — | q.s. | q.s. |

E. pH-Dependently Soluble Release-Controlled Coating Layer

| | | | | | | |
|---|---|---|---|---|---|---|
| Eudragit ® L100-55 | 11.25 | 11.25 | 11.25 | 67.5 | 67.5 | 67.5 |
| Triethyl citrate | 1.13 | 1.13 | 1.13 | 6.75 | 6.75 | 6.75 |
| Talc | 5.63 | 5.63 | 5.63 | 33.75 | 33.75 | 33.75 |
| Methanol* | q.s. | q.s. | q.s. | q.s. | q.s. | q.s. |

*Evaporates during processing.

Manufacturing Procedure:

The seal coating layer, drug layer, sub coating layer, pH-dependently soluble release-controlled coating layer, release-controlled coating layer and encapsulation process for Examples 3, 4 and 5 was similar to the process followed for Examples 1 and 2. Additionally for Example 3, ethyl cellulose was incorporated in the subcoat. In Example 4, a release controlled coating layer comprising a mixture of Eudragit® RSPO and Eudragit® L100-55 was applied over the subcoat, followed by pH-dependently soluble release controlled coating layer. Further, in Example 4, a release controlled coating layer comprising high-viscosity hydroxypropyl methylcellulose was applied over the subcoat, followed by pH-dependently soluble release-controlled coating layer.

### Examples 6-9

### Formulations for Dexlansoprazole 60 mg Capsules

A. Pellet Fraction 1

| | mg/Capsule | | | |
|---|---|---|---|---|
| | Exam- | Exam- | Exam- | Exam- |
| Ingredient | ple 6 | ple 7 | ple 8 | ple 9 |

A. Seal Coating Layer

| | | | | |
|---|---|---|---|---|
| Sugar spheres (25-30 mesh) | 38 | 75 | 38 | 38 |
| Hydroxypropyl methylcellulose, | 2 | 5 | 2 | 2 |
| 5 cps | | | | |
| Methylene dichloride* | q.s. | q.s. | q.s. | q.s. |
| Methanol* | q.s. | q.s. | q.s. | q.s. |

B. Drug Layer 1

| | | | | |
|---|---|---|---|---|
| Dexlansoprazole (amorphous) | 5 | 45 | 11.4 | 5 |
| Magnesium oxide (light) | 3 | 30 | 8 | 3 |
| Polyvinylpyrrollidone K30 | 4 | 25 | 8 | 4 |
| Ethyl cellulose, 4 cps | — | — | — | 2 |
| Methylene chloride* | q.s. | q.s. | q.s. | q.s. |
| Methanol* | q.s. | q.s. | q.s. | q.s. |

C. Subcoating Layer 1

| | | | | |
|---|---|---|---|---|
| Hydroxypropyl methylcellulose, | 7.5 | 7.5 | 7.5 | — |
| 5 cps | | | | |
| Talc | 3 | 3 | 3.6 | — |
| Titanium dioxide | 4.5 | 4.5 | 4.5 | — |
| Isopropyl alcohol* | q.s. | q.s. | q.s. | — |
| Methylene chloride* | q.s. | q.s. | q.s. | — |

D. pH-Dependently Soluble Release-Controlled Coating Layer 1

| | | | | |
|---|---|---|---|---|
| Eudragit ® S 100 | 36.6 | 36.6 | — | — |
| Eudragit ® L100-55 | 9.1 | — | 36 | — |
| Eudragit ® L100 | — | 9.1 | — | — |
| Triethyl citrate | 6.9 | 6.9 | 7 | — |
| Talc | 15 | 15 | 15 | — |
| Methanol* | q.s. | q.s. | q.s. | — |

E. Subcoating Layer 2

| | | | | |
|---|---|---|---|---|
| Hydroxypropyl methylcellulose, | 7.5 | 7.5 | — | — |
| 5 cps | | | | |
| Talc | 3 | 3 | — | — |
| Titanium dioxide | 4.5 | 4.5 | — | — |
| Isopropyl alcohol* | q.s. | q.s. | — | — |
| Methylene chloride* | q.s. | q.s. | — | — |

F. Drug Layer 2

| | | | | |
|---|---|---|---|---|
| Dexlansoprazole (amorphous) | 15 | 15 | 6.6 | 15 |
| Magnesium oxide (light) | 10 | 10 | 4.5 | 10 |
| Polyvinylpyrrollidone K30 | 10 | 10 | 4.5 | 10 |
| Methylene chloride* | q.s. | q.s. | q.s. | q.s. |
| Methanol* | q.s. | q.s. | q.s. | q.s. |

G. Subcoating Layer 3

| | | | | |
|---|---|---|---|---|
| Hydroxypropyl methylcellulose, | 7.5 | 7.5 | — | 7.5 |
| 5 cps | | | | |
| Talc | 3 | 3 | — | 3 |
| Titanium dioxide | 4.5 | 4.5 | — | 4.5 |
| Isopropyl alcohol* | q.s. | q.s. | — | q.s. |
| Methylene chloride* | q.s. | q.s. | — | q.s. |

H. pH-Dependently Soluble Release-Controlled Coating Layer 2

| | | | | |
|---|---|---|---|---|
| Eudragit ® L100-55 | 11.25 | 11.25 | — | 9.1 |
| Eudragit ® S 100 | — | — | — | 36.6 |
| Triethyl citrate | 1.13 | 1.13 | — | 1.13 |
| Talc | 5.63 | 5.63 | — | 5.63 |
| Methanol* | q.s. | q.s. | — | q.s. |

G. Film Coating Layer

| | | | | |
|---|---|---|---|---|
| Hypromellose | — | — | 18.5 | — |
| Polyethylene glycol 6000 (fine | — | — | 15 | — |
| powder) | | | | |
| Talc | — | — | 2.1 | — |
| Titanium dioxide | — | — | 18.5 | — |
| Isopropyl alcohol* | — | — | q.s. | — |
| Methylene chloride* | — | — | q.s. | — |

*Evaporates during processing.

B. Pellet Fraction 2

| | mg/Capsule | | | |
|---|---|---|---|---|
| | Exam- | Exam- | Exam- | Exam- |
| Ingredient | ple 6 | ple 7 | ple 8 | ple 9 |

A. Seal Coating Layer

| | | | | |
|---|---|---|---|---|
| Sugar spheres (25-30 mesh) | 38 | — | 38 | 38 |
| Hydroxypropyl methylcellulose, | 2 | — | 2 | 2 |
| 5 cps | | | | |
| Methylene dichloride* | q.s. | — | q.s. | q.s. |
| Methanol* | q.s. | — | q.s. | q.s. |

B. Drug Layer

| | | | | |
|---|---|---|---|---|
| Dexlansoprazole (amorphous) | 40 | — | 42 | 45 |
| Magnesium oxide (light) | 27 | — | 28 | 30 |
| Polyvinylpyrrollidone K30 | 25 | — | 26 | 30 |
| Methylene chloride* | q.s. | — | q.s. | q.s. |
| Methanol* | q.s. | — | q.s. | q.s. |

C. Subcoating Layer

| | | | | |
|---|---|---|---|---|
| Hydroxypropyl methylcellulose, | 7.5 | — | 9 | 12.5 |
| 5 cps | | | | |
| Talc | 3 | — | 3.5 | 6 |
| Titanium dioxide | 4.5 | — | 4.5 | 6.5 |
| Isopropyl alcohol* | q.s. | — | q.s. | q.s. |
| Methylene chloride* | q.s. | — | q.s. | q.s. |

D. pH-Dependently Soluble Release-Controlled Coating Layer

| | | | | |
|---|---|---|---|---|
| Eudragit ® S100 | 36.6 | — | 36.6 | 36.6 |
| Eudragit ® L100 | 9.1 | — | 9.1 | 9.1 |
| Triethyl citrate | 6.9 | — | 6.9 | 6.9 |
| Talc | 23 | — | 23 | 23 |
| Methanol* | q.s. | — | q.s. | q.s. |

*Evaporates during processing.

Manufacturing Procedure: the seal coating layer, drug layer, sub coating layer, pH-dependently soluble release-controlled coating layer, and encapsulation process for Examples 6, 7, 8, and 9 was similar to the process followed for Examples 1 and 2. Additionally, for Example 8, film coating was done on pellet fraction 1: hypromellose, polyethylene glycol 6000, talc, and titanium dioxide were dissolved in a mixture of isopropyl alcohol and methylene chloride, and this solution was sprayed onto drug layer 2 coated pellets.

### Example 10

### Formulation for Dexlansoprazole 60 mg Capsules

| | mg/Capsule | |
|---|---|---|
| Ingredient | Pellet Fraction 1 | Pellet Fraction 2 |

A. Seal Coating layer

| | | |
|---|---|---|
| Sugar spheres (25-30 mesh) | 38 | 38 |
| Hydroxypropyl methylcellulose, | 2 | 2 |
| 5 cps | | |
| Methylene dichloride* | q.s. | q.s. |
| Methanol* | q.s. | q.s. |

B. Drug Layer

| | | |
|---|---|---|
| Dexlansoprazole (amorphous) | 20 | 40 |
| Magnesium oxide (light) | 13 | 27 |
| Polyvinylpyrrollidone K30 | 13 | 25 |
| Methylene chloride* | q.s. | q.s. |
| Methanol* | q.s. | q.s. |

C. Subcoating Layer

| | | |
|---|---|---|
| Hydroxypropyl methylcellulose, | 7.5 | 10 |
| 5 cps | | |
| Talc | 3 | 5 |
| Titanium dioxide | 4.5 | 7.5 |
| Isopropyl alcohol* | q.s. | q.s. |
| Methylene chloride* | q.s. | q.s. |

D. pH-Dependently Soluble Release-Controlled Coating Layer

| | | |
|---|---|---|
| Eudragit ® S100 | 36.6 | 36.6 |
| Eudragit ® L100-55 | 9.1 | — |
| Eudragit ® L100 | — | 9.1 |
| Triethyl citrate | 1.13 | 2.5 |
| Talc | 5.63 | 8.75 |
| Methanol* | q.s. | q.s. |

*Evaporates during processing.

Manufacturing Procedure: The process for the seal coating layer, drug loading layer, subcoating layer and pH-dependently soluble release-controlled coating layer for Example 10 was similar to the process followed for Examples 1 and 2.

### Examples 11-14

### Formulations for Lansoprazole 30 mg Capsule, Omeprazole 40 mg Capsule, Esomeprazole Magnesium 40 mg Capsule & Rabeprazole Sodium 20 mg Capsules

A. Pellet Fraction 1

| | mg/Capsule | | | |
|---|---|---|---|---|
| | Example | Example | Example | Example |
| Ingredient | 11 | 12 | 13 | 14 |

A. Seal Coating Layer

| | | | | |
|---|---|---|---|---|
| Sugar spheres | 19 | 19 | 19 | 19 |
| (25-30 mesh) | | | | |
| Hydroxypropyl | 1 | 1 | 1 | 1 |
| methylcellulose, 5 cps | | | | |
| Methylene dichloride* | q.s. | q.s. | q.s. | q.s. |
| Methanol* | q.s. | q.s. | q.s. | q.s. |

B. Drug Layer

| | | | | |
|---|---|---|---|---|
| Lansoprazole | 7.5 | — | — | — |
| Omeprazole | — | 10 | — | — |
| Esomeprazole | — | — | 11.15 | — |
| magnesium | | | | |
| Rabeprazole sodium | — | — | — | 5 |
| Magnesium oxide (light) | 5 | 6.6 | 7 | 3.5 |
| Polyvinylpyrrollidone | 5 | 6.6 | 7 | 3.5 |
| K30 | | | | |
| Methylene chloride* | q.s | q.s | q.s | q.s |
| Methanol* | q.s. | q.s. | q.s. | q.s. |

C. Subcoating Layer

| | | | | |
|---|---|---|---|---|
| Hydroxypropyl | 3.75 | 4.5 | 4.7 | 2.5 |
| methylcellulose, 5 cps | | | | |
| Talc | 1.5 | 2 | 2.2 | 1 |
| Titanium dioxide | 2.25 | 2.5 | 2.75 | 2 |
| Isopropyl alcohol* | q.s. | q.s. | q.s. | q.s. |
| Methylene chloride* | q.s. | q.s. | q.s. | q.s. |

D. pH-Dependently Soluble Release-Controlled Coating Layer

| | | | | |
|---|---|---|---|---|
| Eudragit ® L100-55 | 5.62 | 6.0 | 6.5 | 4.5 |
| Triethyl citrate | 0.56 | 0.56 | 0.56 | 0.56 |
| Talc | 2.81 | 3.0 | 2.81 | 2.81 |
| Methanol* | q.s. | q.s. | q.s. | q.s. |

*Evaporates during processing.

B. Pellet Fraction 2

| | mg/Capsule | | | |
|---|---|---|---|---|
| | Example | Example | Example | Example |
| Ingredient | 11 | 12 | 13 | 14 |

A. Seal Coating Layer

| | | | | |
|---|---|---|---|---|
| Sugar spheres | 19 | 19 | 19 | 19 |
| (25-30 mesh) | | | | |
| Hydroxypropyl | 1 | 1 | 1 | 1 |
| methylcellulose, 5 cps | | | | |
| Methylene dichloride* | q.s. | q.s. | q.s. | q.s. |
| Methanol* | q.s. | q.s. | q.s. | q.s. |

B. Drug Layer

| | | | | |
|---|---|---|---|---|
| Lansoprazole | 22.5 | — | — | — |
| Omeprazole | — | 30 | — | — |
| Esomeprazole | — | — | 33.45 | — |
| magnesium | | | | |
| Rabeprazole sodium | — | — | — | 15 |
| Magnesium oxide (light) | 15 | 20 | 22 | 10 |
| Polyvinylpyrrollidone | 15 | 20 | 22 | 10 |
| K30 | | | | |
| Methylene chloride* | q.s | q.s | q.s | q.s |
| Methanol* | q.s. | q.s. | q.s. | q.s. |

C. Subcoating Layer

| | | | | |
|---|---|---|---|---|
| Hydroxypropyl | 11.25 | 15 | 17 | 9.5 |
| methylcellulose, 5 cps | | | | |
| Talc | 4.5 | 5 | 5.5 | 4 |
| Titanium dioxide | 6.75 | 6.75 | 7 | 5.5 |
| Isopropyl alcohol* | q.s. | q.s. | q.s. | q.s. |
| Methylene chloride* | q.s. | q.s. | q.s. | q.s. |

D. pH-Dependently Soluble Release-Controlled Coating Layer

| | | | | |
|---|---|---|---|---|
| Eudragit ® L100-55 | 33.75 | 37 | 38 | 30 |
| Triethyl citrate | 3.37 | 3.5 | 3.8 | 3 |
| Talc | 16.87 | 18 | 18.5 | 15 |
| Methanol* | q.s. | q.s. | q.s. | q.s. |

E. Release-Controlled Coating Layer

| | | | | |
|---|---|---|---|---|
| Eudragit ® RSPO | 18.3 | 20 | 22 | 15 |
| Eudragit ® L100-55 | 4.55 | 6 | 7 | 4 |
| Triethyl citrate | 3.45 | 3.45 | 3.85 | 3 |
| Talc | 11.5 | 12.5 | 12.7 | 11 |
| Methanol* | q.s. | q.s. | q.s. | q.s. |

*Evaporates during processing.

Manufacturing Procedure:

The process for seal coating layer, drug layer, sub coating layer, pH-dependently soluble release-controlled coating layer, release-controlled coating layer and encapsulation process for Examples 11, 12, 13 and 14 was similar to the process followed for Example 1.

### Examples 15-17

### Formulation for Dexlansoprazole 60 mg Capsules

| | mg/Capsule | | | |
|---|---|---|---|---|
| | | Fraction 2 | | |
| | | Example | Example | Example |
| Ingredient | Fraction 1 | 15 | 16 | 17 |

A. Seal Coating Layer

| | | | | |
|---|---|---|---|---|
| Sugar spheres | 28.57 | 43.92 | 43.92 | 43.92 |
| (30-35 mesh) | | | | |
| Polyvinylpyrrollidone | 1.43 | 3.08 | 3.08 | 3.08 |
| K30 | | | | |
| Methanol* | q.s. | q.s. | q.s. | q.s. |

B. Drug Layer

| | | | | |
|---|---|---|---|---|
| Dexlansoprazole | 15 | 45 | 45 | 45 |
| (amorphous) | | | | |
| Magnesium oxide (light) | 10.05 | 30.15 | 30.15 | 30.15 |
| Polyvinylpyrrollidone | 10 | 30 | 30 | 30 |
| K30 | | | | |
| Croscarmellose sodium | 2 | 6 | 6 | 6 |
| (Ac-Di-Sol) | | | | |
| Sodium lauryl sulphate | 2 | 6 | 6 | 6 |
| Sodium Chloride | 2 | 10 | 10 | 10 |
| Methanol* | q.s. | q.s. | q.s. | q.s. |
| Water* | q.s. | q.s. | q.s. | q.s. |

C. Subcoating Layer

| | | | | |
|---|---|---|---|---|
| Hydroxypropyl | 7.1 | 39.18 | 39.18 | 39.18 |
| methylcellulose, 5 cps | | | | |
| Talc | 2.84 | 15.67 | 15.67 | 15.67 |
| Titanium dioxide | 4.26 | 23.51 | 23.51 | 23.51 |
| Isopropyl alcohol* | q.s. | q.s. | q.s. | q.s. |
| Water* | q.s. | q.s. | q.s. | q.s. |

D. pH-Dependently Soluble Release-Controlled Coating Layer 1

| | | | | |
|---|---|---|---|---|
| Hypromellose phthalate | 14.92 | — | 31.56 | 31.56 |
| (HP-55) | | | | |
| Triethyl citrate | 1.49 | — | 3.16 | 3.16 |
| Talc | 5.97 | — | 12.63 | 12.63 |
| Titanium dioxide | 1.49 | — | 3.16 | 3.16 |
| Methanol* | q.s. | — | q.s. | q.s. |
| Acetone* | q.s. | — | q.s. | q.s. |

E. Release-Controlled Coating Layer

| | | | | |
|---|---|---|---|---|
| Eudragit ® RLPO | — | 20.82 | 24.98 | 24.98 |
| Eudragit ® L100-55 | — | 5.2 | 6.24 | 6.24 |
| Triethyl citrate | — | 3.9 | 4.68 | 4.68 |
| Talc | — | 13.01 | 15.61 | 15.61 |
| Methanol* | — | q.s. | q.s. | q.s. |

F. pH-Dependently Soluble Release-Controlled Coating Layer 2

| | | | | |
|---|---|---|---|---|
| Eudragit ® L100-55 | — | 33.24 | — | 39.88 |
| Triethyl citrate | — | 3.32 | — | 3.99 |
| Talc | — | 16.62 | — | 19.94 |
| Methanol* | — | q.s. | — | q.s. |

*Evaporates during processing.

Manufacturing Procedure:

A. Seal Coating of Sugar Spheres (Fractions 1 and 2).

1. Polyvinylpyrrollidone K30 was dissolved in methanol.

2. The solution was sprayed onto sugar spheres using a fluid bed processor (FBP), to achieve a weight gain after drying of 5% for Fraction 1 and 7% for Fraction 2.

B. Drug Loading onto Sugar Spheres (Fractions 1 and 2).

1. Magnesium oxide (light) was homogenized in methanol for 10 minutes.

2. Polyvinylpyrrollidone K30 (PVP K30) was dissolved in methanol.

3. Sodium lauryl sulphate (SLS) was dissolved in water, and sodium chloride was dissolved in water.

4. SLS solution of step 3 was added slowly to PVP K30 solution of step 2 and stirred.

5. Sodium chloride solution of step 3 was added to the solution of step 4 and stirred.

6. Magnesium oxide dispersion of step 1 was added to the solution of step 5 and stirred.

7. Croscarmellose sodium was added to the dispersion of step 6 and stirred.

8. The dispersion of step 7 was transferred to a jacketed vessel equipped with chiller.

9. Dexlansoprazole was added to the dispersion of step 8 and stirred.

10. The dispersion of step 9 is passed through a #100 mesh sieve.

11. The drug dispersion from step 10 was sprayed onto seal coated sugar spheres from step A2.

12. Coated particles were dried at 40° C. until loss on drying (LOD) was less than 5% w/w, measured at 105° C.

C. Subcoating Layer (Fractions 1 and 2).

1. Hydroxypropyl methylcellulose, 5 cps was dissolved in isopropyl alcohol.

2. Talc and titanium dioxide were homogenized in isopropyl alcohol for 10 minutes.

3. Water was slowly added to the HPMC solution of step 1 and stirred to form a clear solution.

4. The dispersion from step 2 was added to the solution of step 1 and stirred.

5. The dispersion of step 4 was passed through a #80 mesh sieve.

6. The dispersion of step 5 was applied to drug coated sugar spheres from step B12, using a fluid bed processor (FBP).

7. The pellets were cured in the FBP for 30 minutes at 40° C.

D. pH-Dependently Soluble Release-Controlled Coating Layer 1 (Fraction 1 of Examples 15, 16, and 17, and Fraction 2 of Examples 16 and 17).

1. Hypromellose phthalate (HP-55) was dissolved in acetone with stirring and methanol was added.

2. Triethyl citrate was added to the solution of step 1 with stirring.

3. Talc and titanium dioxide were homogenized in methanol for 10 minutes.

4. The dispersion of step 3 was added to the solution of step 1 with stirring.

5. The dispersion of step 4 was passed through a #80 mesh sieve.

6. The dispersion of step 5 was sprayed onto 25% of the Fraction 1 subcoated pellets from step C7, to achieve a weight gain after drying of 28±3% w/w, using a FBP with bottom spray.

7. The dispersion of step 5 was sprayed onto 75% of the subcoated pellets from step C7 of Fraction 2 of Examples 2 and 3, to achieve a weight gain after drying of 20±2% w/w, using a FBP with bottom spray.

8. Coated pellets were dried in the FBP at 40° C. product temperature until LOD was 55% w/w at 105° C., measured using a halogen moisture balance.

9. The pellets were cured in the FBP for 1 hour at 40° C.

E. Release-Controlled Coating Layer (Fraction 2).

1. Talc was homogenized in methanol for 10 minutes.

2. Triethyl citrate was dissolved in methanol with stirring.

3. Eudragit® RLPO was added to the solution of step 2 with stirring.

4. Eudragit® L100-55 was added to the solution of step 3 with stirring.

5. Talc dispersion of step 1 was added to the solution of step 4 with stirring.

6. The dispersion of step 5 was passed through a #100 mesh sieve.

7. The dispersion from step 6 was sprayed onto 75% of the pH-dependently soluble release-controlled coated pellets from step D9 of Fraction 2, to achieve a weight gain after drying of 17±2% w/w, using a FBP with bottom spray.

8. Coated pellets were dried in the FBP until LOD of the pellets was 55% w/w, measured at 105° C. using a halogen moisture balance.

9. The pellets were cured in the FBP for 1 hour at 40° C. product temperature.

F. pH-Dependently Soluble Release-Controlled Coating Layer 2 (Fraction 2 of Examples 15 and 17).

1. Talc was homogenized in methanol for 10 minutes.

2. Triethyl citrate was dissolved in methanol.

3. Eudragit® L100-55 was added to the solution of step 2 with stirring.

4. Talc dispersion of step 1 was mixed with the solution of step 3.

5. The dispersion of step 4 was passed through a #100 mesh sieve.

6. The dispersion of step 5 was sprayed onto 75% of the release controlled coated pellets from step E9 of Examples 1 and 3, to achieve a weight gain after drying of 18±2% w/w, using a FBP with bottom spray.

7. Coated pellets were dried in the FBP until LOD was 55% w/w at 105° C., measured using a halogen moisture balance.

8. The pellets were cured in the FBP for 1 hour at 40° C.

G. Encapsulation

1. Coated Fraction 1 and 2 pellets were filled into capsules. Capsules of Example 17 comprising fraction 1 & 2 pellets were packaged in a closed HDPE container with a 3 g molecular sieve desiccant pouch, and stored under accelerated stability testing conditions of 40° C. and 75% RH for 3 months. Samples were analyzed for assay and impurities before, during, and after storage, and the results are shown in Table 3. Samples also are subjected to dissolution testing with the following conditions:

Medium: Acid stage: 0.1N HCl (pH 1.2) for 2 hours, then in
Buffer stage: pH 7.0 phosphate buffer with 5 mM of SLS.

Volume of medium: 900 mL.

Apparatus: USP apparatus 2.

Stirring: 75 rpm.

Duration: 150 minutes.

and the results are shown in Table 4.

TABLE 3

| Parameter (%) | Initial | 1 Month | 2 Months | 3 Months |
|---|---|---|---|---|
| Assay | 101.8 | 101.8 | 98.7 | 100.3 |
| Impurity A | 0.06 | 0.09 | 0.08 | 0.08 |
| Impurity B | ND | 0.03 | 0.03 | 0.04 |
| Impurity C | ND | 0.03 | 0.04 | 0.06 |
| Impurity D | 0.10 | 0.13 | 0.12 | 0.13 |
| Impurity E | 0.05 | 0.06 | 0.06 | 0.03 |
| Impurity F | ND | ND | ND | ND |
| Impurity G | ND | ND | ND | ND |
| Impurity H | 0.03 | 0.04 | 0.04 | ND |
| Total impurities | 0.50 | 0.56 | 0.53 | 0.79 |

ND: Not detected;
NA: Not analyzed.

TABLE 4

Cumulative % of Drug Dissolved

Buffer Stage

| Time | Acid Stage | 45 Minutes | 150 Minutes |
|---|---|---|---|
| Initial | 3.0 | 26.1 | 93.0 |
| 1 Month | 3.8 | 27.5 | 95.6 |
| 2 Months | 3.6 | 25.0 | 91.0 |
| 3 Months | 3.3 | 26.0 | 92.5 |

### Examples 18-19

### Dexlansoprazole 60 mg and 30 mg Capsule Formulations

A. Fraction 1 pellets

mg/Capsule

| | | |
|---|---|---|
| Ingredient | Example 18 | Example 19 |

A. Seal Coating Layer

| | | |
|---|---|---|
| Sugar spheres (25-30 mesh) | 42.85 | 21.42 |
| Polyvinylpyrrolidone (PVP K-30) | 2.15 | 1.07 |
| Methanol* | q.s. | q.s. |

B. Drug Layer

| | | |
|---|---|---|
| Dexlansoprazole (amorphous) | 45 | 22.5 |
| Magnesium oxide (light) | 30 | 15 |
| Sodium chloride | 10 | 5 |
| Polyvinylpyrrolidone (PVP K-30) | 15 | 7.5 |
| Dichloromethane* | q.s. | q.s. |
| Methanol* | q.s. | q.s. |
| Water* | q.s. | q.s. |

C. Subcoating Layer

| | | |
|---|---|---|
| Hydroxypropyl methylcellulose | 35.53 | 17.76 |
| Talc | 7.61 | 3.8 |
| Titanium dioxide | 7.61 | 3.8 |
| Dichloromethane* | q.s. | q.s. |
| Isopropyl alcohol* | q.s. | q.s. |

D. pH-Dependently Soluble Release-Controlled Coating Layer

| | | |
|---|---|---|
| Methacrylic acid copolymer type B | 41.27 | 20.63 |
| (Eudragit ® S 100)# | | |
| Methacrylic acid copolymer type A | 13.77 | 6.88 |
| (Eudragit ® L 100)# | | |
| Triethyl citrate | 5.49 | 2.74 |
| Talc | 27.52 | 13.76 |
| Isopropyl alcohol* | q.s. | q.s. |
| Water* | q.s. | q.s. |

E. Lubrication

| | | |
|---|---|---|
| Talc | 1 | 0.5 |

*Evaporates during processing.
#Quantity given as dry solids weight.

I. Manufacturing Procedure for Fraction 1 Pellets:

A. Seal Coating Layer

1. Polyvinylpyrrolidone was dissolved in methanol.

2. The solution was sprayed onto sugar spheres, using a fluid bed processor (FBP), to achieve a weight gain of 5%.

B. Drug Layer

1. Polyvinylpyrrolidone was dissolved in a mixture of dichloromethane and methanol.

2. Magnesium oxide light was dispersed in a mixture of dichloromethane and methanol.

3. Sodium chloride was dissolved in water and added this solution was added to the dispersion of 2 with stirring for 5 minutes.

4. Dexlansoprazole amorphous was added to the dispersion of 3 with stirring for 15 minutes.

5. The polyvinylpyrrolidone solution of 1 was added to the dispersion of 4 with continuous stirring.

6. Seal coated pellets were coated with the dispersion of 5 using a FBP (bottom spray).

C. Subcoating Layer

1. Hydroxypropylmethylcellulose was dissolved in dichloromethane and isopropyl alcohol.

2. Talc and titanium dioxide were homogenized in dichloromethane and isopropyl alcohol.

3. Dispersion of 2 was added to polymer solution of 1 and stirred.

4. The solution was sprayed onto drug loaded pellets, using a FBP, to achieve a weight gain of 35%.

D. pH-Dependently Soluble Release-Controlled Coating Layer

1. Triethyl citrate was dissolved in isopropyl alcohol.

2. Eudragit® S100 and Eudragit® L100 were added to the solution of 1, with stirring.

3. Water was added with stirring, and stirring was continued to form a clear solution.

4. Talc was added to solution of 3 and continuously stirred throughout the coating process.

5. The dispersion of 4 was sprayed onto subcoated pellets to achieve target weight gain of 45±5%, using a FBP (bottom spray).

6. The pellets were cured in the FBP for 2 hours at 40° C.

7. The dried pellets were sifted through a 16 mesh sieve, and then a 24 mesh sieve, and particles retained on the 24 mesh sieve were used for further processing.

8. Blended the pellets with talc for 15 minutes.

B. Fraction 2 Pellets

mg/Capsule

| | | |
|---|---|---|
| Ingredient | Example 18 | Example 19 |

A. Seal Coating Layer

| | | |
|---|---|---|
| Sugar spheres (25-30 mesh) | 28.5 | 14.25 |
| Polyvinylpyrrolidone (PVP K-30) | 1.5 | 0.75 |
| Methanol* | q.s. | q.s. |

B. Drug Layer

| | | |
|---|---|---|
| Dexlansoprazole (amorphous) | 15 | 7.5 |
| Magnesium oxide (light) | 10 | 5 |
| Polyvinylpyrrolidone (PVP K-30) | 10 | 5 |
| Dichloromethane* | q.s. | q.s. |
| Methanol* | q.s. | q.s. |

C. Subcoating Layer

| | | |
|---|---|---|
| Hydroxypropyl methylcellulose | 11.38 | 5.96 |
| Talc | 2.43 | 1.21 |
| Titanium dioxide | 2.43 | 1.21 |
| Dichloromethane* | q.s. | q.s. |
| Isopropyl alcohol* | q.s. | q.s. |

D. pH-Dependently Soluble Release-Controlled Coating Layer

| | | |
|---|---|---|
| Methacrylic acid copolymer type C | 10.16 | 5.08 |
| (Eudragit ® L 100-55)# | | |
| Triethyl citrate | 1.01 | 0.5 |
| Talc | 5.08 | 2.54 |
| Methanol* | q.s. | q.s. |
| Water* | q.s. | q.s. |

E. Lubrication

| | | |
|---|---|---|
| Talc | 0.5 | 0.25 |

*Evaporates during processing.
#Dry solids weight.

II. Manufacturing Procedure for Fraction 2 Pellets

A. Seal Coating Layer

1. Polyvinylpyrrolidone was dissolved in methanol.

2. The solution was sprayed onto sugar spheres using a FBP, to achieve a weight gain of 5%.

B. Drug Loading

1. Polyvinylpyrrolidone was dissolved in a mixture of dichloromethane and methanol.

2. Magnesium oxide light was dispersed in a mixture of dichloromethane and methanol.

3. Dexlansoprazole amorphous was added to the dispersion of 2 with stirring for 15 minutes.

4. The polyvinylpyrrolidone solution of 1 was added to the dispersion of 3 with continuous stirring.

5. Seal coated pellets were coated with the dispersion of 4 using a FBP (bottom spray).

C. Subcoating Layer

1. Hydroxypropylmethylcellulose was dissolved in dichloromethane and isopropyl alcohol.

2. Talc and titanium dioxide were homogenized in dichloromethane and isopropyl alcohol.

3. Dispersion of 2 was added to polymer solution of 1 and stirred.

4. The dispersion of 3 was sprayed onto drug loaded pellets using a FBP, to achieve a weight gain of 25%.

D. pH-Dependently Soluble Release-Controlled Coating Layer

1. Dissolved Eudragit® L100-55 in methanol.

2. Added water to the solution of 1, with stirring.

3. Added triethyl citrate to the mixture of 2, with stirring.

4. Dispersed talc in the mixture of 3, with stirring.

5. Sprayed the dispersion of 4 onto subcoated pellets using a FBP.

6. Dried coated pellets in the FBP until loss on drying (LOD) of the pellets was 1-3% w/w at 60° C., measured using a halogen moisture balance.

7. Cured the pellets in the FBP for 2 hours at 40° C.

8. Sifted the pellets through a 16 mesh sieve, then through a 20 mesh sieve, used particles retained on the 20 mesh sieve for further processing.

9. Blended the pellets with talc for 15 minutes.

III. Encapsulation

1. Pellets Fraction 1 and Fraction 2 were filled into empty hard capsule shells.

The Fraction 1 pellets of Example 1 were stored unpackaged at the accelerated stability testing conditions 40° C. and 75% RH for 1 week. The samples were analyzed for impurities and results are shown below in Table 5, where values are percentages of the label dexlansoprazole content.

TABLE 5

| Impurity | Initial | Stored |
|---|---|---|
| Impurity A | 0.09 | 0.52 |
| Impurity B | 0.02 | 0.06 |
| Impurity C | 0.03 | 0.09 |
| Impurity D | 0.17 | 0.23 |
| Impurity E | 0.10 | 0.08 |
| Impurity F | 0.02 | 0.04 |
| Impurity G | 0.03 | 0.06 |
| Impurity H | 0.07 | 0.35 |
| Total | 0.72 | 1.59 |

### Examples 20-21

### Dexlansoprazole 60 mg Capsule Formulations

A. Fraction 1 pellets

| | mg/Capsule | |
|---|---|---|
| Ingredient | Example 20 | Example 21 |

A. Seal Coating Layer

| | | |
|---|---|---|
| Sugar spheres (25-30 mesh) | 42.85 | 42.85 |
| Polyvinylpyrrolidone (PVP K-30) | 2.15 | — |
| Hydroxypropyl methylcellulose, 5 cps | — | 2.15 |
| Methanol* | q.s. | q.s. |
| Methylene chloride* | q.s. | q.s. |

B. Drug Layer

| | | |
|---|---|---|
| Dexlansoprazole (amorphous) | 45 | 45 |
| Magnesium oxide (light) | 30 | 30 |
| Polyvinylpyrrolidone (PVP K-30) | 15 | — |
| Hydroxypropyl methylcellulose, 5 cps | — | 15 |
| Methylene chloride* | q.s. | q.s. |
| Methanol* | q.s. | q.s. |

C. Subcoating Layer

| | | |
|---|---|---|
| Hydroxypropyl methylcellulose | 13.5 | 13.5 |
| Talc | 5.4 | 5.4 |
| Titanium dioxide | 8.1 | 8.1 |
| Dichloromethane* | q.s. | q.s. |
| Isopropyl alcohol* | q.s. | q.s. |

D. pH-Dependently Soluble Release-Controlled Coating Layer

| | | |
|---|---|---|
| Methacrylic acid copolymer type B | 37.97 | 37.97 |
| (Eudragit S 100)# | | |
| Methacrylic acid copolymer type A | 12.66 | 12.66 |
| (Eudragit L 100)# | | |
| Triethyl citrate | 5.06 | 5.06 |
| Talc | 25.31 | 25.31 |
| Isopropyl alcohol* | q.s. | q.s. |
| Water* | q.s. | q.s. |

E. Lubrication

| | | |
|---|---|---|
| Talc | 1 | 1 |

*Evaporates during processing.
#Dry solids weight.

B. Fraction 2 pellets

| | mg/Capsule | |
|---|---|---|
| Ingredient | Example 20 | Example 21 |

A. Seal Coating Layer

| | | |
|---|---|---|
| Sugar spheres (25-30 mesh) | 38.1 | 38.1 |
| Polyvinylpyrrolidone (PVP K-30) | 1.9 | — |
| Hydroxypropyl methylcellulose, 5 cps | — | 1.9 |
| Methanol* | q.s. | q.s. |
| Methylene chloride* | q.s. | q.s. |

B. Drug Layer

| | | |
|---|---|---|
| Dexlansoprazole (amorphous) | 15 | 15 |
| Magnesium oxide (light) | 10 | 10 |
| Polyvinylpyrrolidone (PVP K-30) | 10 | — |
| Hydroxypropyl methylcellulose, 5 cps | — | 10 |
| Dichloromethane* | q.s. | q.s. |
| Methanol* | q.s. | q.s. |

C. Subcoating Layer

| | | |
|---|---|---|
| Hydroxypropyl methylcellulose | 5.63 | 5.63 |
| Talc | 2.25 | 2.25 |
| Titanium dioxide | 3.37 | 3.37 |
| Dichloromethane* | q.s. | q.s. |
| Isopropyl alcohol* | q.s. | q.s. |

D. pH-Dependently Soluble Release-Controlled Coating Layer

| | | |
|---|---|---|
| Methacrylic acid copolymer type C | 10.8 | 10.8 |
| (Eudragit ® L 100-55)# | | |
| Triethyl citrate | 1.05 | 1.05 |
| Talc | 5.4 | 5.4 |
| Methanol* | q.s. | q.s. |
| Water* | q.s. | q.s. |

E. Lubrication

| | | |
|---|---|---|
| Talc | 0.5 | 0.5 |

*Evaporates during processing.
#Dry solids weight.

Manufacturing Procedure:

The seal coating, drug layer, sub coating, Fraction 1 coating, Fraction 2 coating, and encapsulation processes for Examples 20 and 21 were similar to the processes followed for Examples 18 and 19.

The Fraction 1 pellets of Examples 20 and 21 were stored unpackaged at the accelerated stability testing conditions 40° C. and 75% RH for 1 week. Samples were analyzed for impurities and data is tabulated in Table 6, where values are percentages of the label dexlansoprazole content.

TABLE 6

| | Initial | | Stored | |
|---|---|---|---|---|
| Impurity | Example 20 | Example 21 | Example 20 | Example 21 |
| Impurity A | 0.38 | 0.55 | 1.01 | 1.44 |
| Impurity B | 0.12 | 0.18 | 0.31 | 0.47 |
| Impurity C | 0.08 | 0.09 | 0.31 | 0.31 |
| Impurity D | 0.20 | 0.18 | 0.27 | 0.48 |
| Impurity E | 0.09 | 0.1 | 0.12 | 0.25 |
| Impurity F | 0.11 | 0.27 | 0.27 | 0.65 |
| Impurity G | 0.11 | 0.12 | 0.15 | 0.37 |
| Impurity H | 0.28 | 0.23 | 0.68 | 0.09 |
| Total | 1.51 | 1.87 | 3.90 | 6.22 |

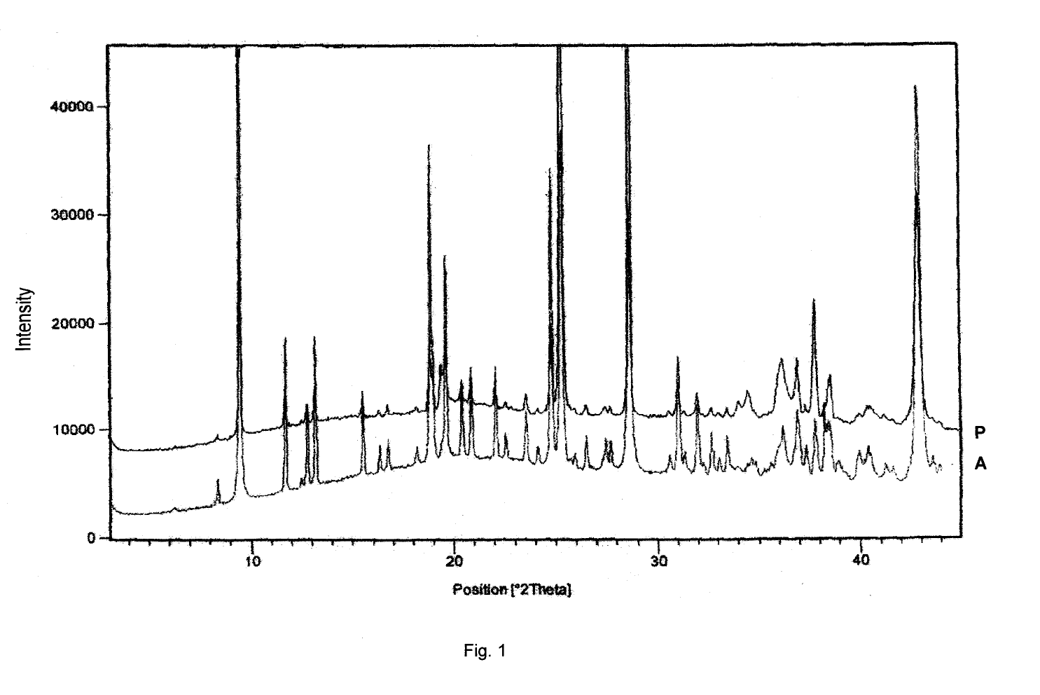

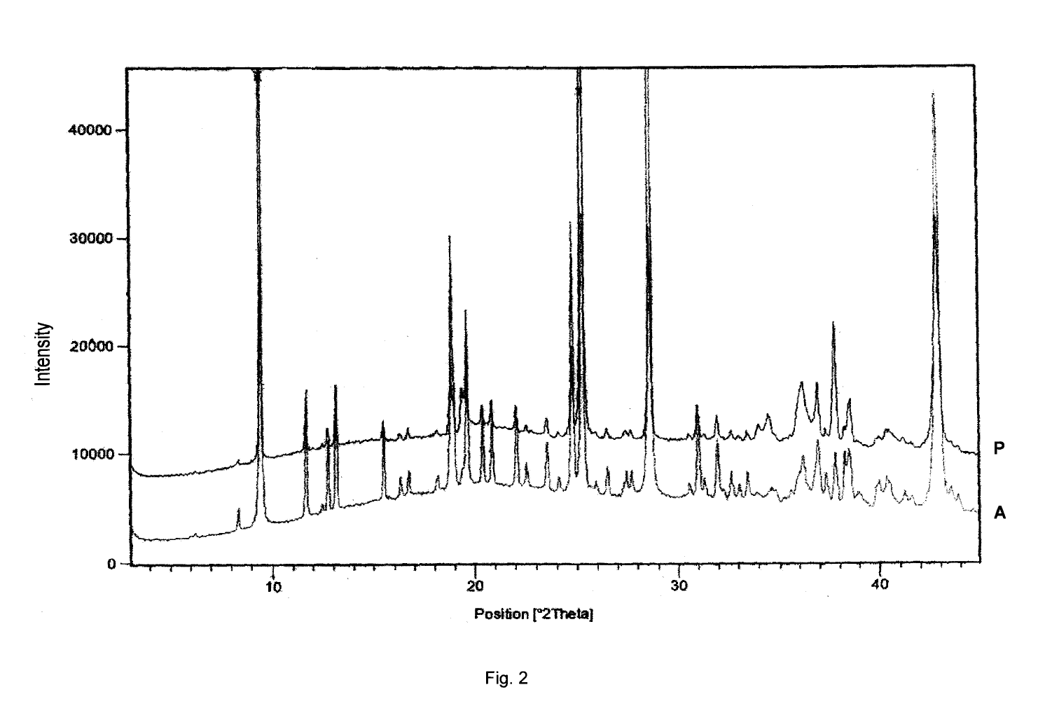

Samples taken before and after storage were subjected to XRPD analysis, using copper Kα radiation. FIG. 1 shows initial comparative XRPD patterns for the formulation prepared according to Example 20, where A represents the formulation (Fraction 2 pellets and Fraction 1 pellets) and P represents a placebo formulation, prepared similarly but without any dexlansoprazole. FIG. 2 shows comparative XRPD patterns after storage, where A represents the formulation (Fraction 1 and Fraction 2) and P represents a placebo formulation. It was observed that dexlansoprazole retains its polymorphic form during storage.

Six of the Example 20 capsules were subjected to dissolution testing with the following conditions:
Medium: 0.1N HCl (pH 1.2) for 2 hours, followed by pH 7.0 phosphate buffer with 0.3% SLS for 3 hours
Volume of medium: 900 mL
Apparatus: USP apparatus 2 (Paddle)
Stirring RPM: 75
   The results are given in Table 7.

TABLE 7

| | Cumulative % of Drug Dissolved | | | | | | |
|---|---|---|---|---|---|---|---|
| Minutes | 1 | 2 | 3 | 4 | 5 | 6 | Mean |

Acid Stage

| | | | | | | | |
|---|---|---|---|---|---|---|---|
| 120 | 2.9 | 2.9 | 1.9 | 2.9 | 3.9 | 1.9 | 2.7 |

Buffer Stage

| | | | | | | | |
|---|---|---|---|---|---|---|---|
| 15 | 14 | 8 | 8 | 9 | 10 | 13 | 10.3 |
| 30 | 26 | 22 | 22 | 24 | 23 | 25 | 23.6 |
| 45 | 46 | 44 | 43 | 46 | 45 | 47 | 45.1 |
| 60 | 57 | 55 | 55 | 57 | 54 | 57 | 55.8 |
| 75 | 66 | 63 | 63 | 65 | 65 | 67 | 64.8 |
| 90 | 77 | 73 | 72 | 75 | 74 | 78 | 74.8 |
| 105 | 86 | 82 | 81 | 83 | 82 | 86 | 83.3 |
| 120 | 92 | 89 | 89 | 90 | 89 | 93 | 90.3 |
| 150 | 95 | 92 | 91 | 94 | 93 | 94 | 93.1 |
| 180 | 94 | 93 | 92 | 93 | 91 | 93 | 92.6 |

### Examples 22-24

### Formulation for Dexlansoprazole 60 mg Capsules

| | mg/Capsule | | | |
|---|---|---|---|---|
| | | Pellet Fraction 2 | | |
| | Pellet | Example | Example | Exam- |
| Ingredient | Fraction 1 | 22 | 23 | ple 24 |

A. Seal Coating Layer

| | | | | |
|---|---|---|---|---|
| Sugar spheres (30-35 mesh) | 28.6 | 43.7 | 43.7 | 43.7 |
| Polyvinylpyrrolidone K30 | 1.4 | 3.3 | 3.3 | 3.3 |
| Methanol* | q.s. | q.s. | q.s. | q.s. |

B. Drug Layer

| | | | | |
|---|---|---|---|---|
| Dexlansoprazole | 15 | 45 | 45 | 45 |
| (amorphous)** | | | | |
| Magnesium oxide (light) | 10 | 30 | 30 | 30 |
| Sodium chloride | 2 | 10 | 10 | 10 |
| Polyvinylpyrrollidone K30 | 10 | 30 | 30 | 30 |
| Sodium lauryl sulphate | 2 | 6 | 6 | 6 |
| Croscarmellose sodium | 2 | 6 | 6 | 6 |
| Water* | q.s | q.s. | q.s. | q.s. |
| Methanol* | q.s. | q.s. | q.s. | q.s. |

C. Subcoating Layer

| | | | | |
|---|---|---|---|---|
| Hydroxypropyl | 7.1 | 39.1 | 25.2 | 25.7 |
| methylcellulose, 5 cps | | | | |
| Talc | 2.8 | 15.7 | 10.1 | 10.3 |
| Titanium dioxide | 4.3 | 23.5 | 15.1 | 15.4 |
| Sodium acetate | — | — | 28.1 | — |
| Mannitol | — | — | — | 27 |
| Water* | q.s. | q.s. | q.s. | q.s. |
| Isopropyl alcohol* | q.s. | q.s. | q.s. | q.s. |

D. pH-Dependently Soluble Release-Controlled Coating Layer 1

| | | | | |
|---|---|---|---|---|
| Hypromellose phthalate | 13.9 | 31.6 | 31.6 | 31.6 |
| (HP 55) | | | | |
| Triethyl citrate | 1.4 | 3.2 | 3.2 | 3.2 |
| Titanium dioxide | 1.4 | 3.2 | 3.2 | 3.2 |
| Talc | 5.5 | 12.6 | 12.6 | 12.6 |
| Acetone* | q.s. | q.s. | q.s. | q.s. |
| Methanol* | q.s. | q.s. | q.s. | q.s. |

E. Release-Controlled Coating Layer

| | | | | |
|---|---|---|---|---|
| Eudragit ® RLPO | — | 22.1 | 22.1 | 22.1 |
| Eudragit ® L100-55 | — | 5.5 | 5.5 | 5.5 |
| Triethyl citrate | — | 4.2 | 4.2 | 4.2 |
| Talc | — | 13.8 | 13.8 | 13.8 |
| Methanol* | — | q.s. | q.s. | q.s. |

F. pH-Dependently Soluble Release-Controlled Coating Layer 2

| | | | | |
|---|---|---|---|---|
| Hypromellose pthalate | — | 54.5 | 54.5 | 54.5 |
| (HP 55) | | | | |
| Dibutyl sebacate | — | 5.45 | 5.45 | 5.45 |
| Talc | — | 21.8 | 21.8 | 21.8 |
| Titanium dioxide | — | 5.45 | 5.45 | 5.45 |
| Acetone* | — | q.s. | q.s. | q.s. |
| Methanol* | — | q.s. | q.s. | q.s. |

G. Lubrication

| | | | | |
|---|---|---|---|---|
| Talc*** | 0.54 | 2.2 | 2.2 | 2.2 |

*Evaporates during processing
**Quantity may vary due to moisture content.
***Present in final formulation in traces.

Manufacturing Procedure:

The seal coating layer, drug layer, sub coating layer, pH-dependently soluble release-controlled coating layer 1, release-controlled coating layer, pH-dependently soluble release-controlled coating layer 2, and encapsulation process for Examples 22-24 was similar to the process followed for Example 17.

Capsules of Example 22 comprising fraction 1 & 2 pellets were packaged in a closed HDPE container with a 3 g molecular sieve desiccant pouch, and stored under accelerated stability testing conditions of 40° C. and 75% RH for 2 months. Samples were analyzed for assay and impurities before, during, and after storage, and the results are shown in Table 8. Samples also are subjected to dissolution testing with the following conditions:

Medium: Acid stage: 0.1 N HCl (pH 1.2) for 2 hours, then in
Buffer stage: pH 7.0 phosphate buffer with 5 mM of SLS.

Volume of medium: 900 mL.

Apparatus: USP apparatus 2.

Stirring: 75 rpm.

Duration: 150 minutes.

and the results are shown in Table 9.

TABLE 8

| Parameter (%) | Initial | 1 Month | 2 Months |
|---|---|---|---|
| Assay | 102.7 | 103.3 | 101.7 |
| Impurity A | 0.04 | 0.07 | 0.09 |
| Impurity B | 0.01 | 0.02 | 0.03 |
| Impurity C | 0.003 | 0.01 | 0.29 |
| Impurity D | 0.10 | 0.12 | 0.13 |
| Impurity E | 0.07 | 0.04 | 0.06 |
| Impurity F | ND | ND | ND |
| Impurity G | ND | ND | ND |
| Impurity H | ND | ND | ND |
| Total impurities | 0.49 | 0.39 | 0.48 |
| XRPD | Amorphous | NA | Amorphous |

ND: Not detected;
NA: Not analyzed.

TABLE 9

Cumulative % of Drug Dissolved

Buffer Stage

| Time | Acid Stage | 45 Minutes | 150 Minutes |
|---|---|---|---|
| Initial | 3.0 | 32.5 | 97.8 |
| 1 Month | 4.0 | 31.5 | 89.3 |
| 2 Months | 1.0 | 32.3 | 90.8 |

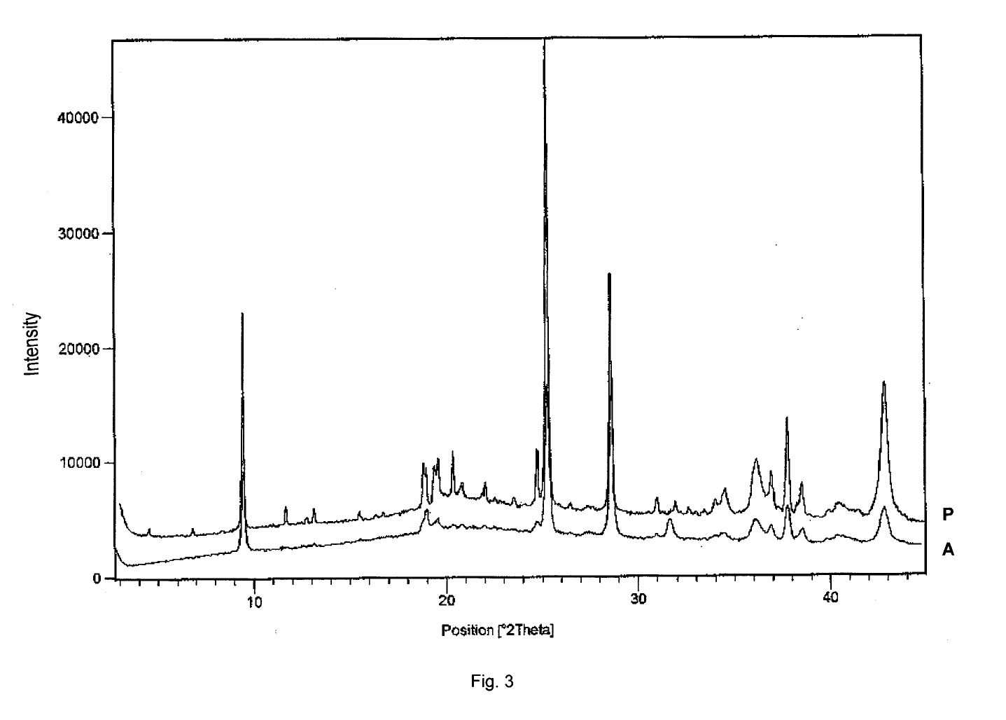

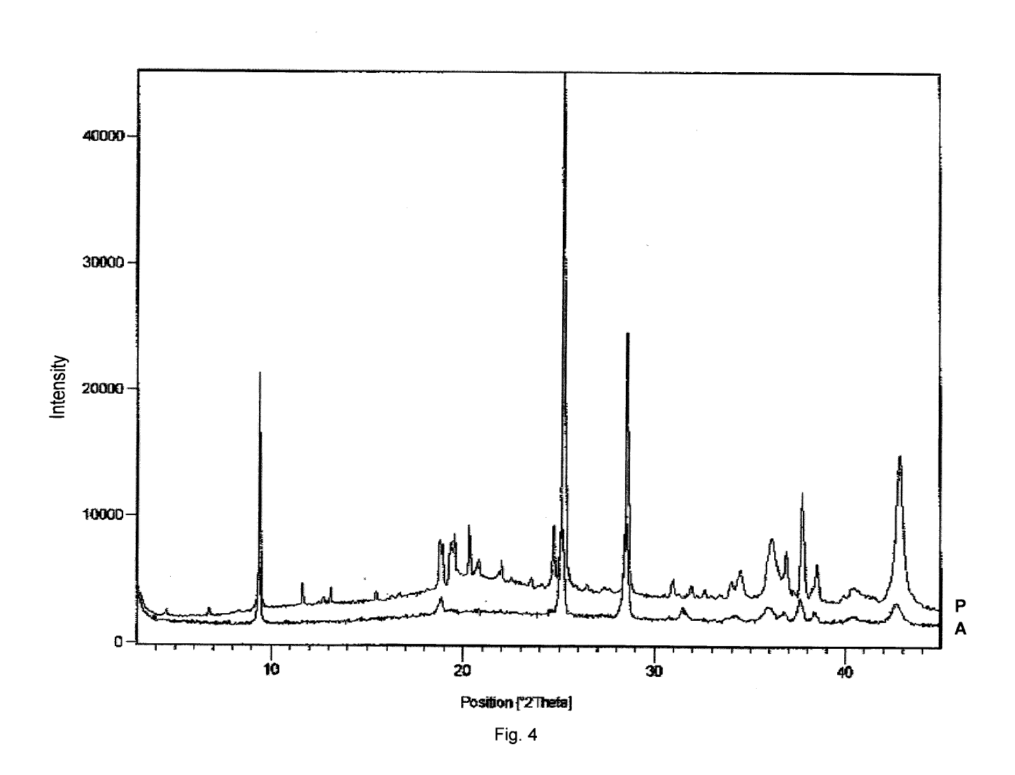

Samples taken before and after storage were subjected to XRPD analysis, using copper Kα radiation. FIG. 3 shows initial comparative XRPD patterns for the formulation prepared according to Example 22, where A represents the formulation (capsule comprising Fraction 2 pellets and Fraction 1 pellets) and P represents a placebo formulation, prepared similarly but without any dexlansoprazole. FIG. 4 shows comparative XRPD patterns after storage at 40° C. and 75% RH for 2 months, where A represents the formulation (capsule comprising Fraction 1 pellets and Fraction 2 pellets) and P represents a placebo formulation. It was observed that dexlansoprazole retains its polymorphic form during storage.

### Example 25

### Formulation for Dexlansoprazole 60 mg Capsules

| | mg/Capsule | |
|---|---|---|
| | | Pellet |
| Ingredient | Pellet Fraction 1 | Fraction 2 |

A. Seal Coating Layer

| | | |
|---|---|---|
| Sugar spheres (30-35 mesh) | 28.6 | 43.7 |
| Polyvinylpyrrolidone K30 | 1.4 | 3.3 |
| Methanol* | q.s. | q.s. |

B. Drug Layer

| | | |
|---|---|---|
| Dexlansoprazole (amorphous) | 15 | 45 |
| Magnesium oxide (light) | 10.1 | 30.2 |
| Sodium chloride | 2 | 10 |
| Polyvinylpyrrollidone K30 | 10 | 30 |
| Croscarmellose sodium | 2 | 6 |
| Sodium lauryl sulphate | 2 | 6 |
| Water* | q.s. | q.s. |
| Methanol* | q.s. | q.s. |

C. Subcoating Layer

| | | |
|---|---|---|
| Hydroxypropyl methylcellulose, 5 cps | 7.1 | 25.7 |
| Talc | 2.9 | 10.3 |
| Titanium dioxide | 4.3 | 15.4 |
| Mannitol | — | 27 |
| Isopropyl alcohol* | q.s. | q.s. |
| Water* | q.s. | q.s. |

D. pH-Dependently Soluble Release-Controlled Coating Layer

| | | |
|---|---|---|
| Hypromellose pthalate (HP 55) | 13.9 | — |
| Triethyl citrate | 1.4 | — |
| Titanium dioxide | 1.4 | — |
| Talc | 5.5 | — |
| Acetone* | q.s. | — |
| Methanol* | q.s. | — |

E. Release-Controlled Coating Layer

| | | |
|---|---|---|
| Ethyl cellulose | — | 3.2 |
| Eudragit ® L100-55 | — | 20 |
| Calcium stearate | — | 13.6 |
| Titanium dioxide | — | 1.4 |
| Talc | — | 3.2 |
| Triethyl citrate | — | 4.1 |
| Ethanol* | — | q.s. |

F. Lubrication

| | | |
|---|---|---|
| Talc | 0.5 | 1.5 |

*Evaporates during processing.

Manufacturing procedure: The process for the seal coating layer, drug layer, subcoating layer, pH-dependently soluble release-controlled coating layer, release-controlled coating layer and lubrication was similar to the process followed for Examples 1 and 2.

1. A pharmaceutical formulation for oral administration, comprising:
(a) a first fraction, comprising:
(i) a core containing a substituted benzimidazole derivative or a pharmaceutically acceptable salt thereof, at least one stabilizer, and at least one pharmaceutically acceptable excipient; and
(ii) a pH-dependently soluble release-controlled coating layer surrounding the core, wherein the pH-dependently soluble release-controlled coating layer dissolves in a pH range of about 5-6; and
(b) a second fraction, comprising:
(i) a core containing a substituted benzimidazole derivative or a pharmaceutically acceptable salt thereof, at least one stabilizer, and at least one pharmaceutically acceptable excipient; and
(ii) a release-controlled coating layer surrounding the core, wherein the release-controlled coating layer comprises one or more pH-independent polymers and one or more pH-dependently soluble release-controlled coating polymers.

2. The formulation according to claim 1, wherein the second fraction comprises one or more pH-dependently soluble release-controlled coating layers, and wherein a pH-dependently soluble release-controlled coating layer dissolves in a pH range of about 5-6.

3. The formulation according to claim 2, wherein the second fraction comprises:
(i) a core containing a substituted benzimidazole derivative or a pharmaceutically acceptable salt thereof, at least one stabilizer, and at least one pharmaceutically acceptable excipient;
(ii) a release-controlled coating layer surrounding the core; and
(iii) a pH-dependently soluble release controlled coating layer surrounding the release-controlled coating layer.

4. The formulation according to claim 2, wherein the second fraction comprises:
(i) a core containing a substituted benzimidazole derivative or a pharmaceutically acceptable salt thereof, at least one stabilizer, and at least one pharmaceutically acceptable excipient;
(ii) a pH-dependently soluble release controlled coating layer surrounding the core; and
(iii) a release-controlled coating layer surrounding the pH-dependently soluble release controlled coating layer.

5. The formulation according to claim 2, wherein the second fraction comprises:
(i) a core containing a substituted benzimidazole derivative or a pharmaceutically acceptable salt thereof, at least one stabilizer, and at least one pharmaceutically acceptable excipient;
(ii) an inner pH-dependently soluble release controlled coating layer surrounding the core;
(iii) a release-controlled coating layer surrounding the inner pH-dependently soluble release controlled coating layer; and
(iv) an outer pH-dependently soluble release controlled coating layer surrounding the release-controlled coating layer.

6. A pharmaceutical formulation for oral administration, comprising:
(a) a first fraction, comprising:
(i) a core containing a substituted benzimidazole derivative or a pharmaceutically acceptable salt thereof, at least one stabilizer, and at least one pharmaceutically acceptable excipient; and
(ii) a pH-dependently soluble release-controlled coating layer surrounding the core, wherein the pH-dependently soluble release-controlled coating layer dissolves in a pH range of about 6-6.5; and
(b) a second fraction, comprising:
(i) a core containing a substituted benzimidazole derivative or a pharmaceutically acceptable salt thereof, at least one stabilizer, and at least one pharmaceutically acceptable excipient; and
(ii) a pH-dependently soluble release-controlled coating layer surrounding the core, wherein the pH-dependently soluble release-controlled coating layer dissolves in a pH range of about 6.5-8.

7. A pharmaceutical formulation for oral administration, comprising:
(a) an inert core;
(b) a first layer containing a substituted benzimidazole derivative or a pharmaceutically acceptable salt thereof, at least one stabilizer, and at least one pharmaceutically acceptable excipient surrounding the core of (a);
(c) a first pH-dependently soluble release-controlled coating layer surrounding the layer of b), wherein the pH-dependently soluble release-controlled coating layer is soluble in a pH range of about 6-8;
(d) a layer containing a substituted benzimidazole derivative or a pharmaceutically acceptable salt thereof, at least one stabilizer, and at least one pharmaceutically acceptable excipient, coated over the pH-dependently soluble release-controlled coating layer of (c); and
(e) a pH-dependently soluble release-controlled coating layer surrounding the layer of (d), wherein the pH-dependently soluble release-controlled coating layer is soluble in a pH range of about 4-6.

8. The formulation according to any of claim 1, 6, or 7, wherein a substituted benzimidazole derivative in either of the fractions independently is rabeprazole, omeprazole, lansoprazole, leminoprazole, pantoprazole, or a single enantiomer thereof, including pharmaceutically acceptable salts thereof.

9. The formulation according to any of claim 1, 6, or 7, wherein a substituted benzimidazole derivative in both fractions is dexlansoprazole, including pharmaceutically acceptable salts thereof.

10. The formulation according to claim 9, wherein dexlansoprazole or a salt thereof is in amorphous form.

11. The formulation according to either of claim 1 or 6, wherein weight ratios of the first fraction to the second fraction are from about 10:90 to about 90:10.

12. The formulation according to claim 7, wherein weight ratios of substituted benzimidazole derivative or salt thereof for the first to the second drug layer are from about 1:99 to about 99:1.

13. The formulation according to either of claim 1 or 6, wherein in vitro release of benzimidazole derivative from the first fraction precedes the release from the second fraction, upon immersion into an aqueous medium having pH at least 5.

14. The formulation according to claim 9 having an in vitro release profile where: no greater than about 10% of dexlansoprazole is released within about 120 minutes after immersion in 500 mL of pH 1.2, 0.1 N hydrochloric acid; then after immersion in 900 mL of pH 7 phosphate buffer with 5 mM SLS, at least about 20% of dexlansoprazole is released within about 90 minutes, and at least about 70% of dexlansoprazole is released within about 200 minutes; using USP apparatus 2 and stirring at 75 rpm.

15. The formulation according to any of claim 1, 6, or 7, wherein a pH-dependently soluble release-controlled coating layer comprises one or more pH-dependently soluble release-controlling polymers.

16. The formulation according to claim 1, wherein a pH-independent polymer is at least one of carbomers, polyamides, polycarbonates, polyalkylenes, polyalkylene glycols, polyalkylene oxides, polyalkylene terepthalates, polyvinyl ethers, polyvinyl esters, polyvinyl halides, polyvinyl acetates, polyglycolides, polysiloxanes, polyurethanes, alkyl celluloses, hydroxyalkyl celluloses, cellulose ethers, cellulose esters, nitrocelluloses, methyl celluloses, ethyl celluloses, hydroxypropyl celluloses, hydroxypropyl methylcelluloses, hydroxyethylcellulose, ethylcellulose, carboxymethylcellulose, polyvinylpyrrolidones, carboxymethylstarch, polyethylene glycols, polyoxyethylenes, poloxamers, polyvinylalcohols, alginates, arabinans, fructans, fucans, galactans, galacturonans, glucans, mannans, xylans, levan, fucoidan, carrageenan, galatocarolose, pectic acid, pectin, amylose, pullulan, glycogen, amylopectin, celluloses, dextran, pustulan, chitin, chitosan, agarose, keratan, chondroitan, dermatan, hyaluronic acid, alginic acid, xanthan gum, guar gum, starchres, aldoses, ketoses, erythrose, threose, ribose, arabinose, xylose, lyxose, allose, altrose, glucose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, neuraminic acid, dextran, cellulose, collagen, albumin, polymers of lactic acid and glycolic acid, polyanhydrides, poly (ortho)esters, polyurethanes, poly (butyric acid), poly (valeric acid), poly (caprolactone), poly (hydroxybutyrate), poly (lactide-co-glycolide), poly (lactide-co-caprolactone) copolymers, alpha-, beta- or gamma-cyclodextrins, dextrin derivatives, ethyl acrylate-methyl methacrylate-trimethyl ammonium ethyl methacrylate chloride copolymers, methyl methacrylate-ethyl acrylate copolymers, cellulose acetates, cellulose butyrates, cellulose diacetates, cellulose triacetates, cellulose propionates, cellulose acetate butyrates, waxes, natural fats, phospholipids, glycerophospholipids, glyceryl palmitostearate, glyceryl behenate, glyceryl monostearate, diethyleneglycol palmitostearate, polyethyleneglycol stearate, polyethyleneglycol palmitostearate, polyoxyethylene-glycol palmitostearate, glyceryl monopalmitostearate, cetyl palmitate, cetyl alcohol, stearic acid, lecithin, cephalins, chitosan, sphingolipids, cholesterol, and polyethylene oxides.

17. The formulation according to any of claim 1, 6, or 7, wherein a pH-dependently soluble release-controlled coating layer comprises one or more of a cellulose acetate phthalate, hydroxypropyl methylcellulose phthalate, polyvinyl acetate phthalate, hydroxypropyl methylcellulose acetate succinate, cellulose acetate trimellitate, hydroxypropyl methylcellulose succinate, cellulose acetate succinate, cellulose acetate hexahydrophthalate, cellulose propionate phthalate, copolymer of methylmethacrylic acid and methyl methacrylate, copolymer of methyl acrylate, methylmethacrylate and methacrylic acid, copolymer of methylvinyl ether and maleic anhydride, zein, shellac, copal collophorium, carboxymethyl ethylcellulose, and co-polymerized methacrylic acid/methacrylic acid methyl ester.

18. The formulation according to any of claim 1, 6, or 7, wherein a stabilizer comprises one or more of polyvinylpyrrolidones, hydroxypropyl celluloses, hydroxyethyl celluloses, hydroxypropyl methylcelluloses, polyvinyl alcohols, carboxymethylcellulose sodium, sugars and sugar alcohols.

19. The formulation according to claim 18, wherein a stabilizer is present in amounts about 0.1 to about 10 percent by weight of the total formulation.

20. The formulation according to any of claim 1, 6, or 7, wherein pharmaceutically acceptable excipients are one or more of diluents, binders, alkalizers, dissolution enhancers, lubricants, glidants, disintegrating agents, antioxidants, surfactants, plasticizers, antiadherents, opacifiers, solvents, colorants, pigments, antifoam agents, and polishing agents.

21. The formulation according to any of claim 1, 6, or 7, containing a drug dissolution enhancer comprising sodium chloride, sodium acetate, potassium chloride, lithium chloride, calcium chloride, magnesium chloride, sodium sulfate, potassium sulfate, sodium carbonate, magnesium sulfate, potassium phosphate, stearyl alcohol, cetyl alcohol, a polyethylene glycol, a poloxamer, a docusate salt, a polyoxyethylene alkyl ether, a polyoxyethylene castor oil derivative, a polysorbate, a polyoxyethylene alkyl ester, sodium lauryl sulfate, a sorbitan monoester, mannitol, glucose, sucrose, xylitol, sorbitol, maltitol; alanine, glycine, and any mixtures of two or more thereof.

22. The formulation according to claim 21, wherein a dissolution enhancer is present in amounts of about 0.1 to about 5 percent by weight of the total formulation.

23. A process for preparing the formulation according to claim 1, comprising:
(a) preparing a first fraction by:
(i) applying a layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying to obtain cores;
(ii) optionally, applying an intermediate coating layer over the cores of step (i); and
(iii) applying a pH-dependently soluble release-controlled coating layer over the core of (i) or the intermediate coating of (ii), wherein the pH-dependently soluble release-controlled coating layer dissolves in a pH range of about 5-6;
(b) preparing a second fraction by:
(i) applying a layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying to obtain cores;
(ii) optionally, applying an intermediate coating layer over the cores of step (i); and
(iii) applying a release-controlled coating layer over the cores of step (i) or the intermediate coating of (ii), wherein the release-controlled coating layer comprises one or more pH-independent polymers and one or more pH-dependently soluble release-controlled coating polymers; and
c) combining the first fraction of (a) and the second fraction of (b), and filling into capsules or compressing into tablets.

24. A process for preparing the formulation according to claim 6, comprising:
(a) preparing a first fraction by:
(i) applying a layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying to obtain cores;
(ii) optionally, applying an intermediate coating layer over the cores of step (i); and
(iii) applying a pH-dependently soluble release-controlled coating layer over the cores of (i) or the intermediate coating of (ii), wherein the pH-dependently soluble release-controlled coating layer dissolves in a pH range of about 6-6.5;
(b) preparing a second fraction by:
(i) applying a layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying to obtain cores;
(ii) optionally, applying an intermediate coating layer over the cores of step (i); and
(iii) applying a pH-dependently soluble release-controlled coating layer over cores of (i) or the intermediate coating of (ii), wherein the pH-dependently soluble release-controlled coating layer dissolves in a pH range of about 6.5-8; and
c) combining the first fraction of (a) and the second fraction of (b), and filling into capsules or compressing into tablets.

25. A process for preparing the formulation according to claim 7, comprising:
(a) applying a first layer of a powder, suspension, dispersion, or solution comprising dexlansoprazole or a pharmaceutically acceptable salt thereof, at least one stabilizer, and at least one pharmaceutically acceptable excipient, onto pharmacologically inert particles, and drying to obtain cores;
(b) optionally, applying an intermediate coating layer over the cores of step (a);
(c) applying a first pH-dependently soluble release-controlled coating layer over cores of (a) or the intermediate coating of (b), wherein the pH-dependently soluble release-controlled coating layer is soluble in a pH range of about 6-8;
(d) optionally, applying an intermediate coating layer over first pH-dependently soluble release-controlled coating layer of step (c);
(e) applying a second layer of a powder, suspension, dispersion, or solution comprising dexlansoprazole or a pharmaceutically acceptable salt thereof, at least one stabilizer, and at least one pharmaceutically acceptable excipient, onto cores of steps (c) or (d);
(f) optionally, applying an intermediate coating layer over the core of step (e);
(g) applying a second pH-dependently soluble release-controlled coating layer surrounding the cores of (e) or the intermediate coating of (f), wherein the pH-dependently soluble release-controlled coating layer is soluble in a pH range of about 4-6; and
(h) filling into capsules or compressing into tablets.

26. A method of treating erosive esophagitis and heartburn associated with non-erosive gastroesophageal reflux disease in a mammal, comprising administering to the mammal an effective amount of the formulation according to any of claim 1, 6, or 7.
